SEQUENCE LISTING (9 sequences)

SEQ ID NO: 1 - protein, 237 aa, Bacillus clausii
ASFLSPLQALKASWSYEGETGPEFWGDLDEAFAACSHGKEQSPINLFFEREQTPKWNWAFSYSEAAFSVENNGHTIQANVENDDAGGLEINGEAYQLTQFHFHTPSEHTIEEASFPMELHLVHANHAGDLAVLGVLMEIGTVHEGIEAVWEVMPEEEGTAEYSISLDPSLFLPESVTAYQYDGSLTTPPCSEGVKWTVLNDTISISATQLHAFRDIYPQNYRPVQELGDREIGFHYH

SEQ ID NO: 2 - protein, 237 aa, Bacillus clausii
ASFLSPLQALKASWSYEGETGPEFWGDLDEAFAACSHGKEQSPINLFFEREQTPKWNWAFSYSEAAFSVENNGHTIQANVENDDAGGLEINGEAYQLTQFHFHTPSEHTIEKTSFPMELHLVHANHAGDLAVLGVLMEIGTVHEGIEAVWEVMPEEEGTAEYSISLDPSLFLPESVTAYQYDGSLTTPPCSEGVKWTVLNDTISISATQLDAFRDIYPQNYRPVQELGDREIGFHYH

SEQ ID NO: 3 - protein, 237 aa, Bacillus clausii
ASFLSPLQAVKASWSYEGETGPEFWGDLDEAFAACSNGKEQSPVNLFFENGPTPKWNWTFSYSEAAFSVENNGHTIQANVENEDAGGLEINGEAYQLTQFHFHTPSEHTIEETSFPMELHLVHANRAGDLAVLGVLMEIGNGHDGIEAVWEVMPEEEGTAEHPISLNPSLFLPESVTAYQYDGSLTTPPCSEGVKWTVLNDTISISAAQLDAFRDIYPQNYRPVQELGDREIGFHYH

SEQ ID NO: 4 - protein, 237 aa, Bacillus clausii
ASFLSPLQALKASWSYEGDTGPEFWGDLDEAFAACSNGKEQSPINLFYDREQTPKWNWAFSYSEAAFSVENNGHTIQANVENEDAGGLEINGEAYQLTQFHFHTPSEHTIEETSFPMELHLVHANHAGDLAVLGVLMEIGNDHEGIEAVWEVMPEEEGTAEYSISIDPSLFLPESVTAYQYDGSLTTPPCSEGVKWTVLNDTISISATQLDAFRAIYPQNYRPVQELGDREIGFHYH

SEQ ID NO: 5 - protein, 237 aa, Bacillus clausii
ASFLSPLQAVKASWSYEGETGPEFWGDLDEAFAACSNGKEQSPVNLFFENGPTPKWNWTFSYSEAAFSVENNGHTIQANVENDDAGGLEINGEAYQLTQFHFHTPSEHTIEETLFPMELHLVHANHAGDLAVLGVLMEIGNGHGGIETVWEIMPEEEGTAEHPISLNPSLFLPENVTAYQYDGSLTTPPCSEGVKWTVLNDTISISAAQLDAFSDIYPQNYRPVQELGDREIGFHYH

SEQ ID NO: 6 - protein, 237 aa, Bacillus clausii
ASFLSPLQALKASWSYEGDTGPEFWGDLDEAFAACSNGKEQSPINLFYDREQTPKWNWAFSYSEAAFSVENNGHTIQANVENEDAGGLEINGEAYQLTQFHFHTPSEHTIEETSFPMELHLVHANHAGDLAVLGVLMEIGNDHEGIEAVWEVMPEEEGTAEYSISIDPSLFLPESVTAYQYDGSLTTPPCSEGVKWTVLNDTISISATQLDAFRDIYPQNYRPVQELGDREIGFHYH

SEQ ID NO: 7 - protein, 237 aa, Bacilllus clausii
ASFLSPLQALKASWSYEGETGPEFWGDLDEAFAACSNGKEQSPINLFYDREQTSKWNWAFSYSEAAFSVENNGHTIQANVENEDAGGLEINGEAYQLIQFHFHTPSEHTIEETSFPMELHLVHANHAGDLAVLGVLMEMGNDHEGIEAVWEVMPEEEGTAAYSISLDPNLFLPESVTAYQYDGSLTTPPCSEGVKWTVLNDTISISETQLDAFRDIYPQNYRPVQELGDREIGFHYH

SEQ ID NO: 8 - protein, 250 aa, Bacillus Halodurans
PSTEPVDEPSETHEETSGGAHEVHWSYTGDTGPEHWAELDSEYGACAQGEEQSPINLDKAEAVDTDTEIQVHYEPSAFTIKNNGHTIQAETTSDGNTIEIDGKEYTLVQFHFHIPSEHEMEGKNLDMELHFVHKNENDELAVLGVLMKAGEENEELAKLWSKLPAEETEENISLDESIDLNALLPESKEGFHYNGSLTTPPCSEGVKWTVLSEPITVSQEQIDAFAEIFPDNHRPVQPWNDRDVYDVITE

SEQ ID NO: 9 - protein, 224 aa, Caminibacter mediatlanticus
TWSYSGKTGPEYWGDLKKEYQMCKIGKNQSPIDIKTNSTQTFNTNLKPFKIKYLGKGYEVINNGHTIKVKTEGKNCVRIDGIKFKLAQLHFHTPSENTINGKHFPMEAHYVHLDKNGNITVLAVMYKIGKENKSLNKMLAVLPTKVGEENKVMGNLNPMELLPKNKAYYRFNGSLTTPPCSEGVRWIVFKTPVEISQAQYEKMHAVMGTNNRPVQPINARVILK

(19) United States
(12) Patent Application Publication
(10) Pub. No.: US 2011/0223650 A1
(43) Pub. Date: Sep. 15, 2011

(54) Modular Membrane Reactor and Process for Carbon Dioxide Extraction

(75) Inventors: Paria Saunders, Knightdale, NC (US); Sonja Salmon, Raleigh, NC (US); Martin Borchert, Hilleroed (DK); Louis Patrick Lessard, Wake Forest, NC (US)

(73) Assignee: Novozymes A/S, Bagsvaerd (DK)

(21) Appl. No.: 13/056,472

(22) PCT Filed: Jul. 30, 2009
(86) PCT No.: PCT/US09/52194

§ 371 Date: Jan. 28, 2011

Related U.S. Application Data
(60) Provisional application No. 61/085,030, filed on Jul. 31, 2008.

(51) Int. Cl. A62D 3/02; C12M 1/00

(52) U.S. Cl. 435/266; 435/283.1

(57) ABSTRACT

The present invention relates to a reactor and a process suitable for extracting carbon dioxide from carbon dioxide-containing gas stream. The reactor is based on a two module system where absorption occurs in one module and desorption occurs in the other module. The carbon dioxide extraction may be catalyzed by carbonic anhydrase.

### STATEMENT OF GOVERNMENT INTEREST

This invention was made with Government support under Prime Contract No. DE-FC26-07NT43084 awarded by the Department of Energy. The Government has certain rights in this invention.

### FIELD OF THE INVENTION

The present invention relates to reactors and processes capable of separating carbon dioxide (CO₂) from a mixed gas using separate modules for absorption and desorption of the carbon dioxide. The extraction of CO₂ may be facilitated using a carbonic anhydrase. Mixed gases are, e.g., CO₂-containing gases such as flue gas from coal or natural gas power plants, biogas, landfill gas, ambient air, synthetic gas or natural gas or any industrial off-gas containing carbon dioxide.

### BACKGROUND OF THE INVENTION

Carbon dioxide (CO₂) emissions are a major contributor to the phenomenon of global warming. CO₂ is a by-product of combustion and it creates operational, economic, and environmental problems. CO₂ emissions may be controlled by capturing CO₂ gas before emitted into the atmosphere. There are several chemical approaches to control CO₂ emissions. One approach is to pass the CO₂ through an aqueous liquid containing calcium ions, allowing the CO₂ to precipitate as CaCO₃. Preferred techniques for capturing CO₂ gas from combustion processes are ones in which the product of the capture process is CO₂ in the form of a gas that can be compressed and transported to storage sites, or for useful purposes. The most well-established technique for extracting CO₂ from a gaseous feed and capturing the extracted CO₂ gas for use or storage is absorption of CO₂ into amine. The major drawback to this approach is the high energy consumption overall (and especially in the desorption step), slow processes, oxidation and degradation of amines and use of ecological questionable or toxic or corrosive compounds, such as amines.

Solutions capable of separating carbon dioxide from gas streams and which do not need amines or heating to regenerate the absorbing capacity of the solution are known in the art. These solutions are based on the ability of CO₂ gas to diffuse into an aqueous liquid that contains alkaline compounds such as alkaline salt solutions, where dissolved CO₂ is hydrated to produce an equilibrium of carbonic acid, bicarbonate and carbonate ions. Biological (e.g., carbonic anhydrase) catalysts are able to increase the rate of the CO₂ hydration reaction. It is reported that carbonic anhydrase can catalyze the conversion of CO₂ to bicarbonate at a very high rate (turnover numbers up to 10⁵ molecules of CO₂ per second are reported). In order to capture the CO₂ the ions may be precipitated as a carbonate salt and removed from the liquid in the solid form, or converted back into CO₂ (dehydration) and removed from the liquid in the gaseous form.

Reactors and processes for extracting CO₂ from gases, such as combustion gases or respiration gases, using liquid membranes have been described. For example, reactors including hollow fiber membranes containing a liquid film are described in Majumdar et al., 1988, AlChE 1135-1145; U.S. Pat. No. 4,750,918; U.S. Pat. No. 6,156,096; WO 04/104160. In the prior art such hollow fiber membrane-based designs have been termed hollow fiber liquid membranes (HFLM) and the CO₂ separation devices based on these have been termed hollow fiber contained liquid membrane (HFCLM) permeators. A common feature of HFCLM permeators is that the hollow fibers enclosing the feed and sweep gas streams are near (i.e., “tightly packed” or “immediately adjacent”) to one another and they are enclosed in a single rigid treatment chamber to form one complete permeator. In such a design, a liquid surrounds the shell side of the tightly packed feed and sweep hollow fibers. Because the distance between the outside wall of one hollow fiber is very close to adjacent hollow fibers the thickness of the liquid layer between them is thin, like a membrane, and the composition of the liquid only allows certain components to pass, hence the term “liquid membrane” has been used to describe the liquid surrounding the hollow fibers. Contained liquid membrane permeators where the liquid film is sandwiched between two structural support membranes have also been described in the art (Cowan et al., 2003, Ann. NY Acad. Sci. 984: 453-469); this design essentially functions in the same way as the HFCLM. Other prior art describes the CO₂ absorption-stripping behaviour of a concentrated aqueous solution of a non-volatile amine using hollow fiber membrane modules (Kosaraju et al., 2005, Ind. Eng. Chem. Res. 44:1250-1258).

Reactors for extracting CO₂ from gases, such as combustion gases or respiration gases, using membranes in combination with carbonic anhydrases have been described. In one case, CO₂ is removed from a gaseous stream by passing the gaseous stream through a gas diffusion membrane into solution where conversion to carbonic acid is accelerated by passing the CO₂ solution over a matrix that contains carbonic anhydrase and adding a mineral ion to cause precipitation of the carbonic acid salt (U.S. Pat. No. 7,132,090). In another case, reactors utilizing contained liquid membranes in combination with carbonic anhydrase are described in U.S. Pat. No. 6,143,556, WO 2004/104160, Cowan et al., 2003, Ann. NY Acad. Sci. 984: 453-469; and Trachtenberg et al., 2003, SAE international Conference on Environmental Systems Docket number 2003-01-2499. In these cases, the CO₂ desorption step takes place in the same enclosed treatment chamber as the adsorption step.

### DRAWINGS

FIG. 1 is a schematic presentation of a serial module hollow fiber membrane reactor. The numbers represent the following features: 1. Carbon Dioxide (CO₂) tank; 2. Nitrogen (N₂) tank; 3. Mass flow controllers (MFC); 4. Carrier liquid reservoir; 5. Liquid pump; 6. Pressure gauge; 7. Absorption module; 8. Desorption module; 9. Feed gas; 9a feed gas entering absorption module; 10. Scrubbed gas; 11. Mass flow meter (MFM); 12. Gas sampling valve; 13. Gas chromatograph with thermal conductivity detector (GC-TCD); 14. CO₂-containing gas in; 15. Scrubbed gas out; 16. Liquid in; 17. Liquid out; 18. Sweep stream in; 19. Sweep stream+CO₂ out.

FIG. 2 is a schematic presentation of a hollow fiber membrane module. The numbers represent the following features: 1. Module casing; 2. carrier liquid flow (Lumen flow); 3. Gas in; 4. Gas out; 5. Individual hollow fibers; 6. Fiber wall; 7. Fiber pores; 8. Lumen of the hollow fibers. In the hollow fiber membrane module presented in the figure the liquid and gas phase can also be reversed in that case 2=gas flow; 3=carrier liquid in; 4=carrier liquid out.

FIG. 3.A. illustrates a reactor with two parallel connected absorption modules where the outlet gas (scrubbed gas) from the first absorption module (7a) is passed to the second absorption module (7b). The carbon rich carrier liquid (4a) from the first absorption module is passed on to a first desorption module (8a) and the carrier liquid from the second absorption module is passed on a to a second desorption module (8b) and the lean carrier liquid (4b) from the two desorption modules are collected in a carrier liquid reservoir (4), which supply the first and second absorption modules. B. Illustrates two parallel connected absorption modules as in A, the desorption modules are connected in series 8a receive carrier liquid from 7a and potentially from 7b (alternatively the carrier liquid from 7b is passed to 8b). The carrier liquid from 8a is passed to 8b which passes the carrier liquid to the reservoir. The numbers represent the following features: 4. Carrier liquid reservoir; 4a. Rich carrier liquid passing from the first absorption module to the first desorption; 4b. Lean carrier liquid passing from the desorption modules to the liquid reservoir; 5. Liquid pump; 7a. First absorption module; 7b. Second absorption module; 8a. First desorption module 8b. Second desorption module; 9a Feed gas entering first absorption module; 9b. Feed gas entering second absorption module; 10a. Scrubbed gas exiting first absorption module; 10b. Scrubbed gas exiting second absorption module; 18. Sweep stream in; 19. CO₂-enriched sweep stream out

FIG. 4.A. illustrates a schematic side view of a spiral-wound membrane module. The carrier liquid enters the module at 2 in a zone where it flows directly into the liquid channel formed by 3. Carrier liquid flows through 3 towards 6 where carrier liquid enters the collection tube through pores 4 and travels along the collection tube to exit the module at 7. Gas enters the module at 8, is transported through the gas channel 9, and exits the module at 10. The numbers represent the following features: 1. module housing; 2. carrier liquid in; 7. carrier liquid out; 8. gas in; 10. gas out. B. Illustrates a schematic cross-sectional view of A where the spiral-wound membrane design includes two gas-permeable membranes “X” and “Y.” The numbers represent the following features: 1. module housing; 3. spacer material that forms a liquid channel; 4. pore in the collection tube wall; 5. collection tube wall; 6. carrier liquid out zone; 9. spacer material that forms a gas channel; 11. CO₂-permeable flat sheet membrane “X”; 12. CO₂-permeable flat sheet membrane “Y”.

FIG. 5 is a schematic presentation of a hollow fiber membrane module set up in desorption mode. The numbers represent the following features: 1. Nitrogen (N₂) tank; 2. Mass flow controller (MFC); 3. Carrier liquid reservoir; 4. Liquid pump; 5. Desorption module; 6. Waste solution; 7. Sweep stream; 7a. Sweep stream entering desorption module; 8. Sweep stream+CO₂ exiting the module; 9. Mass flow meter (MFM); 10. Gas sampling valve; 11. Gas chromatograph with thermal conductivity detector (GC-TCD); 12. Carrier liquid in; 13. Carrier liquid out; 14. pH monitoring device.

### DETAILED DESCRIPTION OF THE INVENTION

One aspect of the invention is a modular reactor suitable for extraction of CO₂ from a CO₂-containing gas. The reactor comprises at least one absorber module and at least one desorber module, and a circulating carrier liquid. In the absorber module CO₂ is absorbed by a chemical or physical solvent and/or the CO₂ is hydrated to bicarbonate (this module is also termed the hydration module). The CO₂ is absorbed in such a way that it can be transported from one module to another by means of the carrier liquid. In the desorber module CO₂ is released from the chemical or physical solvent and/or dehydration of the bicarbonate in the carrier liquid to CO₂ takes place (this module is also termed the dehydration module). Each module comprises at least one CO₂-permeable membrane, e.g., in the form of a hollow fiber membrane, a flat sheet membrane or a spiral-wound membrane. The carrier liquid is circulated through the absorber module to the desorber module and from the desorber module to the absorber module. The modules are preferably connected to a liquid supply (not necessarily part of the circuit), to secure maintenance of the carrier liquid in particular evaporated carrier liquid may need to be re-supplied in order to maintain the system in an overall steady-state.

Preferably, the reactor is an enzyme-based reactor (bioreactor). A preferred enzyme for the bioreactor is a carbonic anhydrase belonging to EC 4.2.1.1. Preferably the carrier liquid recirculates through the reactor.

Another aspect of the present invention is a process for extraction of CO₂ from a CO₂-containing gas by passing a CO₂-containing gas through at least one absorber module where a carrier liquid is enriched in CO₂ (through dissolution, hydration, or chemical reaction of CO₂ with the carrier liquid), allowing the enriched carrier liquid to pass from the absorber module to at least one desorber module, where CO₂ is extracted from the carrier liquid. Preferably, a reactor of the present invention is used for this process. Encompassed in the present invention are reactor designs and processes where the carrier liquid can pass through two or more absorber modules before entering a desorber module. It is also encompassed that the carrier liquid can pass through two or more desorber modules before re-entering an absorber module. Furthermore, the carrier liquid can pass through at least two sequential groups of absorber and desorber modules (where a group means at least one) before the carrier liquid optionally is circulated to a reservoir.

### DEFINITIONS

The term “absorption module” or “absorber module” as used in the present invention describes a carrier liquid containing structure where CO₂ is absorbed by a chemical or physical solvent and/or CO₂ is hydrated to carbonic acid, bicarbonate and/or carbonate. An absorption module of the present invention may comprise a gas-permeable hollow fiber membrane, a gas-permeable flat sheet membrane stack, and/or a gas-permeable spiral-wound membrane. Preferably the gas permeable membranes in the modules are microporous. An absorption module where CO₂ is hydrated to bicarbonate may also be termed a hydration module. When it is stated that CO₂ is hydrated to bicarbonate it is understood that an equilibrium or steady state among carbonic acid, bicarbonate and carbonate is established.

The term “desorption module” or “desorber module” as used in the present invention describes a structure where a) CO₂ is released from the chemical or physical solvent, and/or b) carbonic acid, bicarbonate and/or carbonate is dehydrated to CO₂. A desorption module of the present invention may comprise a gas-permeable hollow fiber membrane, a gas-permeable flat sheet membrane stack, and/or a gas-permeable spiral-wound membrane. Preferably the gas permeable membranes in the modules are microporous. A desorption module where bicarbonate is dehydrated to CO₂ may also be termed a dehydration module. When it is stated that bicarbonate is dehydrated to CO₂, it is understood that CO₂ is formed from the equilibrium or steady state concentrations of carbonic acid, bicarbonate and carbonate established in the carrier liquid.

The term “carbonic anhydrase activity” or “CA activity” is defined herein as an EC 4.2.1.1 activity which catalyzes the inter-conversion between carbon dioxide and bicarbonate [CO₂+H₂O⇄HCO₃⁻+H⁺]. One unit of CA activity is defined after Wilbur [1 U=(1/t_{c})−(1/tᵤ)×1000] where U is units and t_{c} and tᵤ represent the time in seconds for the catalyzed and uncatalyzed reaction, respectively (Wilbur, 1948, J. Biol. Chem. 176: 147-154).

The term “carrier liquid” as used in the present invention describes a liquid, capable of absorbing CO₂, that flows through at least one absorption module to at least one desorption module. The carrier liquid may either be circulated directly from the absorption module(s) to the desorption module(s) or it can be passed from the absorption module through one or more intermediate processing steps, e.g., a carrier liquid reservoir for pH adjustment, or further absorption modules, before the carrier liquid is passed through the desorption module. The carrier liquid leaving the absorption module will generally be enriched in carbon, e.g., in the form of dissolved CO₂, chemically reacted CO₂, bicarbonate, carbonic acid and/or carbonate salt. The terms “CO₂-lean” and “CO₂-rich” carrier liquid are terms used in the present invention to describe the relative amount of carbon (in the form of dissolved CO₂, chemically reacted CO₂, bicarbonate, carbonic acid and/or carbonate salt) present in the carrier liquid as it circulates through the process. As used herein, the term “CO₂-lean carrier liquid” generally refers to carrier liquid entering an absorption module. The term “CO₂-rich carrier liquid” generally refers to a carrier liquid entering a desorption module. It is understood that the term “CO₂-lean carrier liquid” can also be applied to carrier liquid exiting a desorption module, and the term “CO₂-rich carrier liquid” can also be applied to carrier liquid exiting an absorption module.

The term “CO₂-containing gas” is used to describe gaseous phases which may at 1 atm pressure contain at least 0.001% CO₂, preferably at least 0.01%, more preferably at least 0.1%, more preferably at least 1%, more preferably at least 5%, most preferably 10%, even more preferred at least 20%, and even most preferably at least 50% CO₂. The term CO₂-containing gas and mixed gas is used interchangeably. A CO₂-containing gaseous phase is, e.g., raw natural gas obtainable from oil wells, gas wells, and condensate wells, syngas generated by the gasification of a carbon containing fuel (e.g., methane) to a gaseous product comprising CO and H₂, or emission streams from combustion processes, e.g., from carbon based electric generation power plants, or from flue gas stacks from such plants, industrial furnaces, stoves, ovens, or fireplaces or from airplane or car exhausts. A CO₂-containing gaseous phase may alternatively be from respiratory processes in mammals, living plants and other CO₂ emitting species, in particular from green-houses. A CO₂-containing gas phase may also be off-gas, from aerobic or anaerobic fermentation, such as brewing, fermentation to produce useful products such as ethanol, gas generated from landfills, or from the production of biogas. A CO₂-containing gaseous phase may alternatively be a gaseous phase enriched in CO₂ for the purpose of use or storage. The above described gaseous phases are also intended to cover multiphase mixtures, where the gas co-exists with a certain degree of fluids (e.g., water or other solvents) and/or solid materials (e.g., ash or other particles).

The term “CO₂-containing liquids” are any solution or fluid, in particular aqueous liquids, containing measurable amounts of CO₂, preferably at one of the levels mentioned above. CO₂-containing liquids may be obtained by passing a CO₂-containing gas or solid (e.g., dry ice or soluble carbonate containing salt) into the liquid. CO₂-containing liquids may also be compressed CO₂ liquid (that contains contaminants, such as dry-cleaning fluid), or supercritical CO₂, or CO₂ solvent liquids, like ionic liquids. A bicarbonate enriched carrier liquid (CO₂-rich carrier liquid) obtained from the hydration module is also considered to be a CO₂-containing liquid.

The term “CO₂ enriched gas” is used to describe a gas where the CO₂ content has been increased compared to the CO₂ content of the sweep stream entering the desorption module. Preferably, the CO₂ content when measured at 1 atm pressure is increased by 20%, more preferably by 30%, 40%, 50%, 60%, 70%, more preferably by 80%, more preferably by 85%, even more preferably by 90%, even more preferably by 95%, even more preferably by 98%, even most preferably by 99% and most preferred by 100% compared to the CO₂ content of the entry sweep gas. The CO₂ enriched gas of the present invention exits from the dehydration module either on the basis of a driving force such as a pressure difference, or heat, or pH, or agitation (such as vibration), or sweep gas or by diffusion.

The term “CO₂ extraction” is to be understood as a removal of CO₂ from a CO₂-containing gas. Such an extraction may be performed from one medium to another, e.g., gas to liquid, liquid to gas, or gas to liquid to gas.

The term “CO₂ extraction” is to be understood as a reduction or removal of carbon from a CO₂-containing medium such as a CO₂-containing gas. Such an extraction may be performed from one medium to another, e.g., gas to liquid, liquid to gas, gas to liquid to gas, liquid to liquid or liquid to solid, but the extraction may also be the conversion of CO₂ to bicarbonate, carbonate or carbonic acid within the same medium or the conversion of bicarbonate to CO₂ within the same medium. The term CO₂ capture is also used to indicate extraction of CO₂ from one medium to another or conversion of CO₂ to bicarbonate/carbonate or conversion of bicarbonate/carbonate to CO₂.

The term “feed gas” is the gas entering the absorption module. The feed gas is also termed mixed gas or flue gas or gas in.

The term “gas-side” when used in relation to a membrane describes the surfaces of the structural membrane which is primarily in contact with a gas phase. It can also be described as the surface of the membrane which is facing away from the carrier liquid.

The term “liquid-side” when used in relation to a membrane describes the surfaces of a structural membrane that are in contact with a carrier or core liquid of the present invention.

The term “liquid reservoir” describes means for supplying liquid to the reactor and/or process of the present invention ensuring process control, e.g., in terms of flow rate, volume and composition, of the liquid circulating in the system of the present invention. The liquid reservoir may either be in the form of a vessel physically containing a liquid supply. Preferably, such a vessel is integrated into the reactor. Alternatively, the liquid may be supplied by an external source of liquid which is supplied to the system, e.g., via a pipeline. The term liquid reservoir may be used interchangeably with the term liquid supply.

The term “membrane” as used in the present invention describes a solid, gas permeable, sheet-like (the length and width dimensions are larger than the thickness) structure acting as a boundary or partition between two phases, e.g., between a gas and a liquid phase. The sheet-like structure can be shaped to fit the physical requirements of a reactor. For example, the membrane can be produced as a hollow fiber tube or as a flat sheet, or as spiral-wound sheets or in other appropriate shapes. Preferably, a membrane used in the reactors of the present invention are selectively permeable to CO₂, meaning that the membrane allow CO₂ to pass through the membrane easier than other gases, e.g., O₂, N₂, SO₂, etc. The membranes of the present invention may function as structural membranes, e.g., allowing a liquid film to be formed between/within them. In the prior art such liquid films are also termed liquid membranes, e.g., supported liquid membrane, contained liquid membrane or hollow fiber contained liquid membrane. In the present invention a liquid enclosed by one or more structural membranes is termed a “core liquid”. Core liquids of the present invention can also be termed carrier liquids. A gas permeable membrane of the present invention may be microporous. Preferably, the size of the pores is small enough to prevent carrier liquid from passing completely through the pores due to the surface tension of the liquid.

The term “scrubbed gas” is used to describe a gas leaving the absorption module. The term scrubbed gas is in particular used to describe a gas which contains less CO₂ than the feed gas that entered into the absorption module. Preferably, the reduction in CO₂ in the scrubbed gas when compared to the feed gas is least 10%, preferably at least 20%, 30%, 40%, 50%, more preferably at least 60%, 70%, more preferably at least 80%, more preferably at least 85%, even more preferably 90%, most preferably 95%, even more preferred at least 98%, and even most preferably at least 99%, and most preferred 100%.

The term “sweep stream” is used to describe a gas stream or a reduction of pressure applied to the desorption module (e.g., vacuum) which allows for increased extraction of CO₂ from the module.

The term “Syngas” or “synthesis gas” is used to describe a gas mixture that contains varying amounts of carbon monoxide and hydrogen generated by the gasification of a carbon containing fuel (e.g., methane or natural gas) to a gaseous product with a heating value. CO₂ is produced in the syngas reaction and must be removed to increase the heating value.

### Bioreactors and Processes

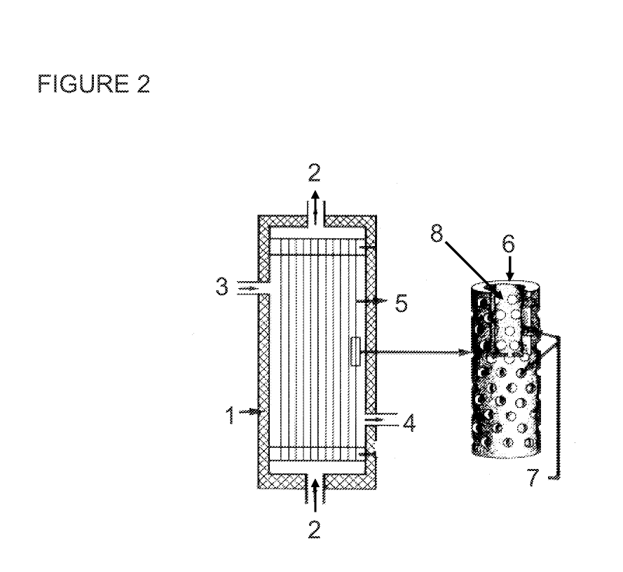

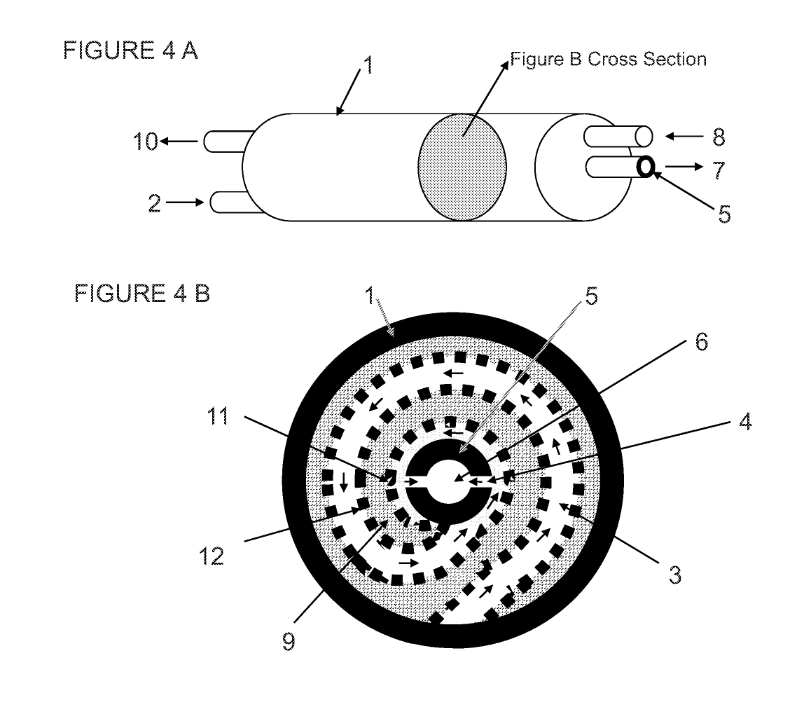

The reactor of the present invention is based on a process in which a mixed gas stream (e.g., containing nitrogen and carbon dioxide) is brought into contact with a gas-liquid interface in a first reactor module. Once the CO₂ is passed from the gas into the liquid equilibrium between bicarbonate, carbonic acid, dissolved CO₂, and carbonate will be established in the liquid phase. The gas-liquid interface in such a reactor module can, e.g., be provided by a carrier liquid enclosed by a structural gas permeable membrane. Preferably, the gas permeable membrane has a high surface area to facilitate a large area of gas-liquid contact allowing as much gaseous CO₂ to interact with the core liquid as possible. A large surface area can, e.g., be obtained by using a porous gas permeable membrane. Preferably, the gas permeable membrane is hydrophobic in order to prevent the core liquid from passing across the membrane from the liquid side to the gas side. Suitable structural membranes include polypropylene gas exchange membranes (e.g., Celgard PP-2400), PTFE (poly-tetrafluorethylene (Teflon), e.g., PTFE-Gore-Tex®), Nafion membranes, poly(4-methyl-1-pentene), polyimide, polyolefin (including polypropylene), polysulfone, silicone, or co-polymers and/or composites of these, zeolites, chytosan, polyvinylpyrollindine and cellulose acetate. These membranes may optionally be coated or laminated to improve resistance to liquids passing across the membrane. Suitable commercially available membranes are for example. Superphobic® Contactors, Membrana GmbH, Wuppertal, Germany for degassing low surface tension fluids, such as liquids containing surfactants. Alternative membranes are composed of hollow-fiber membrane mats or arrays, e.g., Celgard X40-200 or X30-240. Combinations of different membrane shapes or properties (e.g., thickness, porosity, chemical composition) may be used in the present invention to optimize the CO₂ extraction process. In one design of the reactor, the carrier liquid can pass through the lumen (or core) of the hollow fibers, while the feed gas (in the case of the absorber module) passes on the shell (or outside surface) of the hollow fibers (see FIG. 2). The core liquid is preferably continuously re-supplied through a reservoir of carrier liquid solvent. The position of the liquid and gas phase in the hollow fibers may also be reversed, such that the feed gas (in the case of the absorber module) passes through the hollow fibers (in the core) and the carrier liquid passes along the shell (or outside surface) of the hollow fibers. Another design is a spiral-wound membrane where at least two flat sheet membranes separated by spacers are positioned around a collection pipe (see FIG. 4). Another type of design useful in a reactor of the present invention is a spiral-wound membrane design where parallel hollow fibers separated by spacers are positioned around a collection pipe. In the present invention, the collection pipe can transport carrier liquid from one module to another. Another design is the flat sheet membrane stack. The membrane containing modules of the present invention may be selected from any of the above described membrane shapes. In a preferred embodiment the membrane containing module is a hollow fiber membrane and/or a flat sheet membrane stack and/or a spiral wound membrane. The absorption module may be composed of one membrane structure whereas the desorption module is composed of another membrane structure. If there is more than one absorption module these need not be composed of the same membrane structures, the same goes for multiple desorption modules.

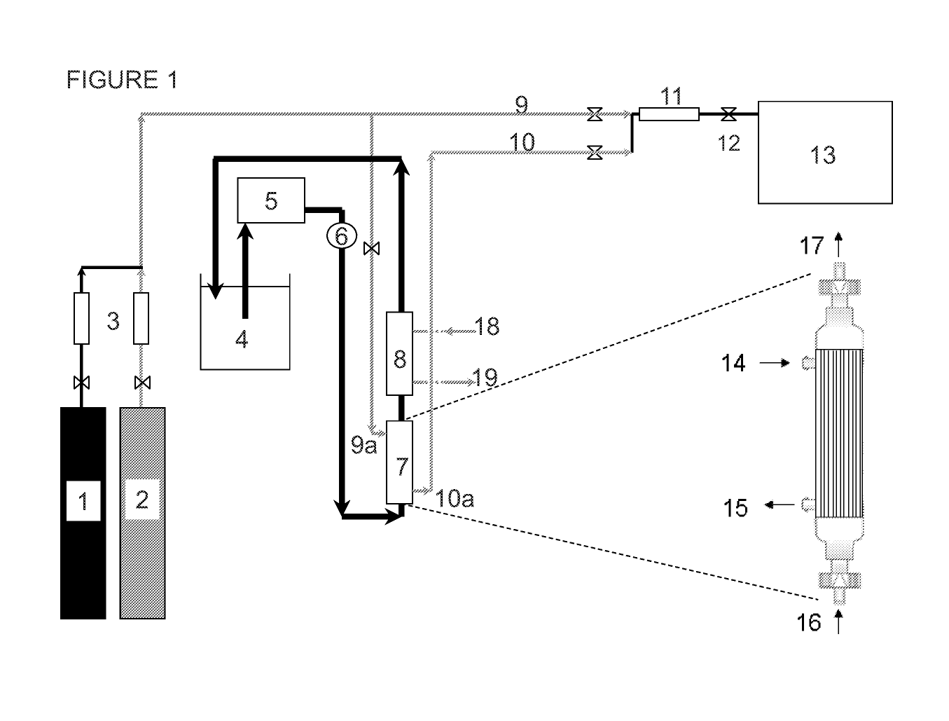

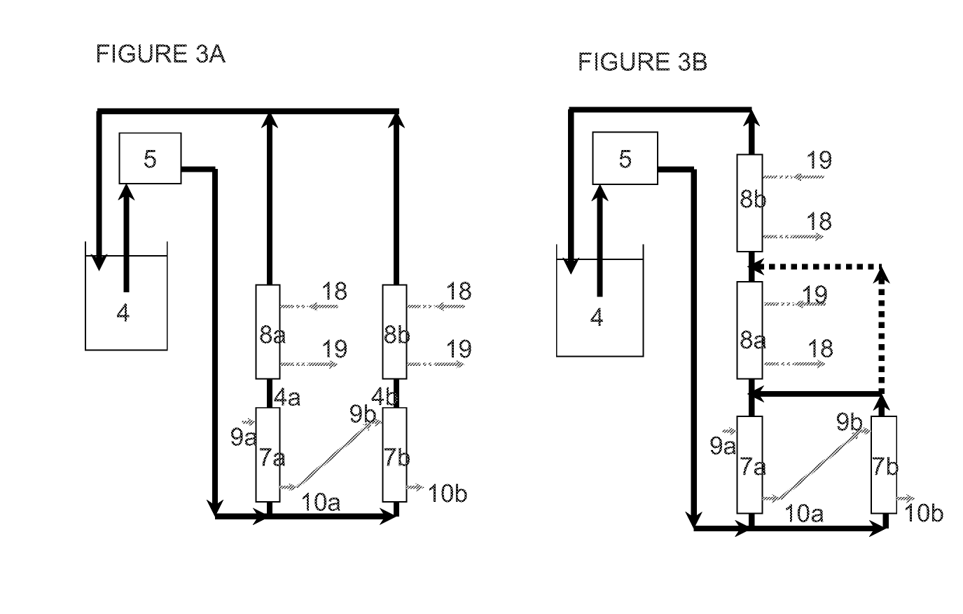

Following the absorption, hydration or dissolution or chemical reaction of CO₂ in the first reactor module the carrier liquid, which is now enriched in bicarbonate or CO₂ in dissolved or chemically reacted form, flows to a second reactor module. The second module is distinctly separated from the first module. In the second module the opposite reaction of converting bicarbonate in the liquid into CO₂ takes place or CO₂ is released from the chemical or physical solvent with which it has reacted. This process of converting bicarbonate in the liquid into CO₂ involves dehydration of the bicarbonate and the second module is, therefore, termed the dehydration module in cases where this reaction occurs. Similarly, the first module is termed the hydration module in the event where CO₂ is converted into bicarbonate in this module. The modules may be connected by a serial flow (illustrated in FIG. 1) or a parallel flow (illustrated in FIG. 3). Reactor designs with more than two (multiple) modules are also contemplated by the present invention. There may, e.g., be one hydration module and two dehydration modules or two hydration and two dehydration modules, or two hydration modules and one dehydration module. These are mere examples and do not exclude other combination of modules.

The CO₂ may pass in and out of the liquid phase by diffusion (pressure aided) and/or the transfer may be aided by an enzyme or a chemical or physical solvent that have affinity toward CO₂. A preferred enzyme is carbonic anhydrase. Since carbonic anhydrase reacts specifically with dissolved CO₂, it favors the movement of gaseous CO₂ into the fluid in the absorption module by accelerating the reaction of the dissolved CO₂ and water to form carbonic acid which dissociates into bicarbonate, and carbonate, thereby removing CO₂ rapidly and allowing the dissolution of more CO₂ from the feed gas stream into the water to a greater extent than would occur only by diffusion. Likewise carbonic anhydrase will catalyze the reverse reaction in the desorption/dehydration module converting bicarbonate into CO₂ which will be released from the carrier liquid in the desorption/dehydration module. The CO₂ can be collected from the desorption/dehydration module either in a sweep stream or by application of a vacuum, i.e., pressure difference or the CO₂ may just diffuse across the gas permeable membrane in the module. The selectivity and speed of the reaction can be increased by adding carbonic anhydrase to the reactor. In a preferred embodiment of the present invention at least one of the modules contains carbonic anhydrase and preferably both modules contain carbonic anhydrase. Preferred chemical solvents are, e.g., amine-based solvents or aqueous ammonia or amino acids which absorb CO₂ through a chemical reaction. Physical CO₂ solvents absorb CO₂ without causing a chemical reaction. Preferably the physical solvent has selectivity for carbon dioxide, including solvents such as, but not limited to, glycerol, polyethylene glycol, polyethylene glycol ethers, polyethylene glycol dimethyl ethers, Selexol™ (Union Carbide), water, refrigerated methanol, NMP, or glycerol carbonate.

The biocatalyst carbonic anhydrase or a chemical catalyst used to facilitate the CO₂ absorption into the carrier liquid may either be in solution in the carrier liquid circulating through the reactor and/or may be immobilized on the membrane in the modules, e.g., by cross-linking and/or by affixing a gel or polymer matrix containing the carbonic anhydrase or chemical onto the membrane. Alternatively, the carbonic anhydrase or chemical may be immobilized on a solid support contained within the core liquid of the liquid membrane. In a preferred embodiment the biocatalyst (e.g., carbonic anhydrase) is present in the bioreactor together with a CO₂ absorbing chemical (e.g., amine-based solvents such as piperazine or MEA) and/or a physical solvent (e.g., polyethylene glycol ethers, or Selexol™)

The reactor design of the present invention provides an increased flexibility. For example, it is easy to replace, add or remove modules from the system, i.e., for maintenance or to increase or decrease the gas-liquid surface area which can be regulated through the number of modules. The modules are less complex compared to CLM designs. The use of separate absorber and desorber modules allows for separate temperature control which allows for use of CO₂ absorber chemicals, such as amines, which require high temperatures in the desorption module to release the CO₂. The use of carbonic anhydrase improves the efficiency of CO₂ extraction both in the presence or absence of other CO₂ absorber chemicals in the carrier liquid.

Furthermore, by letting absorption and desorption occur in separate modules parameters influencing these steps can be optimized separately. For example, it is possible to increase the temperature in one module compared to the other module, such that the temperature of the desorption module is different from the temperature of the absorption module, e.g., by supplying the module with increased temperature means for heating, e.g., a heating cap or an electric current or a steam source, preferably of low pressure. In one embodiment of the present invention the desorption module(s) is maintained at a temperature which is at least 5° C., preferably 10° C., more preferably 15° C., more preferred 20° C., even more preferably 30° C. higher than the temperature in the absorption module. In an embodiment of the present invention the absorption module(s) is maintained at a temperature which is at least 5° C., preferably 10° C., more preferably 15° C., more preferred 20° C., even more preferably 30° C. higher than the temperature in the desorption module. The temperature at which the reactor is operated will be dependent on the temperature of the inlet gas. The process temperature in the bioreactor or the feed gas (e.g. flue stream from a combustion process) temperature may be between 0° C. and 120° C. For hot feed gases the process temperature is between 40° C. and 100° C., or between 45° C. and 110° C., or between 50° C. and 90° C., or between 55° C. and 80° C. or between 60° C. and 75° C., or between 65° C. and 70° C. For other applications where the feed gas temperature is lower the process temperature may be considerably lower, e.g., between 5° C. and 40° C. The temperature can be regulated by cooling or heating the mixed gas stream before it enters the reactor or by supplying heat to desired parts of the reactor. In a bioreactor the temperature is preferably adapted to the optimum temperature of the enzyme present in the reactor. Normally mammalian, plant and prokaryotic carbonic anhydrases function at 37° C. or lower temperatures. However, WO 2008/095057, US 2006/0257990, US 2008/0003662 and U.S. application no. 61220636 describe heat-stable carbonic anhydrases. In a preferred embodiment of the present invention a heat-stable carbonic anhydrase is applied in a bioreactor of the present invention.

The pressure may also be regulated for the individual modules. In one embodiment of the present invention the desorption module(s) is maintained at a pressure which is higher than the pressure in the absorption module. In another embodiment of the present invention the absorption module(s) is maintained at a pressure which is higher than the pressure in the desorption module. The feed gas may be at atmospheric pressure, or at pressures above or below atmospheric pressure. Selective solubility of CO₂ in the carrier liquid causes extraction of CO₂ from the feed gas into the carrier liquid in the absorber. In the desorber, CO₂ is released from the carrier liquid by introducing a pressure difference. For example, a lower partial pressure of CO₂ in the desorber gas phase compared to that in the feed gas can be achieved by applying vacuum in the desorber, this lowers the solubility of CO₂ in the carrier liquid and functions as a desorption driving force. The CO₂ in the desorber may also be driven into the gas phase by applying heat, (e.g., via a reboiler or steam) or by applying a sweep gas. When heat energy is used alone to drive desorption such as is common in monoethanol amine-based CO₂ extraction processes the temperature in the desorber is typically greater than 100° C. (e.g., 120° C.). The pressure difference can be applied in combination with heat and/or a sweep gas to generate a combined driving force in the desorption module. If heat energy is combined with pressure reduction to drive desorption the temperature in the desorber can be lowered. For example, if vacuum is used in the desorber compared to atmospheric pressure in the absorber the temperature of the desorber can be reduced to 70° C. A pressure difference (e.g. vacuum), a sweep gas stream or a low pressure steam can be applied to the desorption module by one or more inlet zones. When heat and/or vacuum are applied in the system, one or more condensers are preferably used to remove water vapor from exiting gas streams. Condensed water vapor can optionally be recycled back to the carrier liquid to maintain liquid levels in the system by balancing for evaporation that may occur across the membrane(s).

A pressure difference between the absorber and the desorber can be established/occur when the pressure of the feed gas passing through the absorber is higher than the pressure of the gas phase in the desorber. In some cases, such as for natural gas upgrading, the gas pressure in the absorber is higher than in the desorber and the gas pressures in both the absorber and the desorber may be above atmospheric pressure. In other cases, the gas pressure in the absorber is above atmospheric pressure and the gas pressure in the desorber is at or below atmospheric pressure (i.e. equal to or less than 100 kPa). Alternatively, a pressure difference between the absorber and the desorber can be established/occur when the pressure of the feed gas (such as a coal-fired post-combustion flue gas) passing through the absorber is approximately at atmospheric pressure and the pressure of the gas phase in the desorber is below atmospheric pressure. In one embodiment of the present invention, the total gas pressure difference between the absorber and the desorber is at least 20 kPa, preferably at least 35 kPa, More preferably at least 50 kPa, even more preferably at least 65 kPa, and even more preferred at least 80 kPa. Preferably the pressure in the desorber is lower than the pressure in the absorber.

Regeneration of CO₂ using low pressure (e.g. between 2 to 90 KPa, preferably between 14 and 55 kPa) at temperatures between 45° C. and 110° C., or between 50° C. and 90° C., or between 55° C. and 80° C. or between 60° C. and 75° C., or between 65° C. and 70° C. in the desorber together with a heat stable carbonic anhydrase as described in WO 2008/095057, US 2006/0257990, US 2008/0003662 and U.S. application no. 61220636 is a further embodiment of the present invention. The vacuum carbonate process has been described for CO₂ extraction in US 2007/0256559 and disclosed in combination with carbonic anhydrase (Lu et al., DOE Project No. DE-F026-08NT0005498, NETL CO2 Capture Technology for Existing Plants R&D Meeting, Mar. 24-26, 2009, Pittsburgh, Pa.). In this illustration, atmospheric pressure power plant flue gas contacts aqueous potassium carbonate and carbonic anhydrase in the absorber module at temperatures in the range 40° C. to 60° C., where carbonic anhydrase is said to improve the rate of CO₂ hydration to bicarbonate in the carrier liquid. The CO₂-rich carrier liquid is pumped to a desorber column (“stripper”) where CO₂ is released from the carrier liquid by a combination of low pressure (e.g. 14-55 KPa) and the application of heat (e.g. 50° C. to 70° C.) obtained by directly injecting low pressure, low quality exhaust steam from a low pressure steam turbine of the power plant. The Caminibacter mediatlanicus carbonic anhydrase described in Example 1 of U.S. application no. 61220636 is especially suitable for use in the described modified vacuum carbonate process because Caminibacter carbonic anhydrase can tolerate temperatures both in the absorber and the desorber, meaning that, unlike other known carbonic anhydrases that would be inactivated by the temperature in the desorber, Caminibacter carbonic anhydrase could tolerate the temperature in the desorber, allowing it to circulate along with the carrier liquid through both absorption and desorption stages of the process.

An aspect of the present invention is a bioreactor for extracting carbon dioxide from a gas phase, where said reactor comprises the following elements:
a) at least one absorption module comprising at least one gas permeable membrane and a gas inlet zone and a gas outlet zone and a carrier liquid,
b) at least one desorption module comprising at least one gas permeable membrane in fluid communication with said absorption module such that the carrier liquid from said absorption module can circulate to the desorption module and optionally be returned to the absorption module, said desorption module further comprises a gas outlet zone, optionally one or more gas inlet zone; and
c) one or more carbonic anhydrases (EC 4.2.1.1); and
d) optionally, means for heating the desorption module; and
e) optionally, a source for reducing the pressure in the desorption module, for example a vacuum source connected with the desorption module.

The means for heating the desorption module may be a low pressure steam connected with the desorption module. The low pressure steam may also function as a desorption driving force in line with or together with the decreased pressure. When more than one desorption module is used, the same driving force may be applied to all modules or a different desorption driving force may be applied in different desorption modules, e.g. vacuum applied in one desorption module, steam or heat in a second desorption module and a sweep gas in a third desorption module. Alternatively, the conditions of the desorption driving force can be changed from one desorption module to the next, e.g. one vacuum condition in one desorption module, and a different (e.g. lower) vacuum condition applied in a second desorption module.

The absorption and desorption rates of CO₂ are dependent on the pH in the carrier liquid. The pH of the carrier liquid upon entry into the absorption module (lean carrier liquid) is preferably above pH 7, more preferably above pH 8, more preferably between 8 and 12, more preferably between 8 and 10.5, more preferably between 8.5 and 10, even more preferably between 9 and 9.5. When the pH of the carrier liquid in the absorption module is above pH 8 the hydration of CO₂ to carbonic acid (which immediately dissociates in water) will result in a decrease of the pH in the carrier liquid. The pH of the carrier liquid will, therefore, be lower upon entry into the desorption module. In order to recirculate carrier liquid through the system, it is preferred to be able to return the pH of the carrier liquid to the target pH before the carrier liquid re-enters the absorption module. The target pH of the carrier liquid (as measured at room temperature, e.g., 20-25° C.) is at least pH 6.5, more preferably above pH 7, more preferably above pH 7.5, more preferably above pH 8, even more preferably between pH 8 and 12, or within one of the other pH ranges mentioned above. In a preferred embodiment of the present invention the reactor is equipped with means for regulating pH in the carrier liquid. This can be performed in several ways. One way is to add an alkaline substance to the carrier liquid, e.g., in the reservoir using automatic pH adjustment equipment such as an automatic titrator. The alkaline substance preferably has a similar composition (e.g., concentration of solvent, ionic strength, amount of carbonic anhydrase, etc.) as the carrier liquid circulating in the system and can be added at any time before absorption for adjustment of pH. Similarly a neutral to acidic substance can be added to the carrier liquid any time before desorption. Alternatively, two carrier liquid supplies can be prepared one with more alkaline pH (e.g., pH 8 to 12) and one with more neutral to acidic pH (e.g., pH 4 to 7). By addition of a more alkaline carrier liquid supply before absorption, the absorption reaction will be more efficient. Likewise by addition of more neutral to acidic carrier liquid supply before desorption, the desorption step will be more efficient. Preferably, the carrier liquid added will not change the total concentration of carrier liquid circulating through the system. When carbonic anhydrase is included in the carrier liquid, more enzyme can be added to the circulating carrier liquid via a liquid supply. This liquid supply can be the same as or different than the liquid supply used to adjust pH. Preferably, the liquid supply containing carbonic anhydrase is added in such a way that the stable pH range of the enzyme is not exceeded, either by being too low or too high. Extra carrier liquid can be removed from the system if needed. Another way to regulate pH in the process is by changing the conditions in the absorber or desorber modules. For example, by applying a driving force to increase the removal of CO₂ from the desorption module; this shifts the equilibrium among the carrier liquid components towards desorption thereby increasing the pH of the carrier liquid. The modularity of the present reactor system allows for such a desorption based regulation of the pH. This can, e.g., be done by supplying a sweep stream to the desorption module. The sweep stream could be a substantially CO₂-free gas, e.g., helium, argon or nitrogen, or a sweep gas where the partial pressure of CO₂ in the sweep gas as it enters the dehydration module is lower than when it exits the module. The sweep stream could also be a vacuum allowing the extraction of substantially pure CO₂. In a preferred embodiment of the present invention the desorption module is supplied with a gas inlet and a gas-outlet in order to facilitate the application of a sweep stream to the desorption module.

The process of CO₂ desorption can be broken down in to two distinct steps. The first step, converting bicarbonate ion to aqueous CO₂ (Equation 1), a process that is facilitated by carbonic anhydrase,

HCO₃⁻ [symbol] OH⁻+CO₂(aq). (1)

The second step involves the transport of CO₂ from the aqueous phase to the gas phase (Equation 2).

CO₂(aq) [symbol] CO₂(g) (2)

From Equation (1) we get the following equilibrium relation

$${K}_{eq} = \frac{[ {CO}_{2} ( aq ) ] [ {OH}^{-} ]}{[ {HCO}_{3}^{-} ]} , \qquad ( 3 )$$

where K_{eq} is an equilibrium coefficient, and we believe that the transport of CO₂ from the aqueous phase to the gas phase can be described by equation 4,

r_{CO}_{_{2(g)}}=k_{aq→g}[CO₂(aq)]−k_{g→aq}P_{CO}_{_{2(g)}} .(4)

where r_{CO}_{_{2(g)}} is the net rate (moles/time) at which CO₂ is transferred from the aqueous phase to the gas phase; k_{aq→g} is the mass transfer coefficient for CO₂ moving from the aqueous to the gas phase; and k_{g→aq} is the mass transfer coefficient of the reverse process. For a process where the concentration of CO₂ in the gas phase is very low we may neglect the second term on the right hand side of Equation 4 resulting in Equation 5,

r_{CO}_{_{2(g)}}=k_{aq→g}[CO₂(aq)]. (5)

Example 4 illustrates that the use of surfactants increases the rate of mass transfer from the aqueous to the gas phase (r_{CO}_{_{2(g)}}) by increasing the mass transfer coefficient k_{aq→g}, thereby shifting the equilibrium of equation 2 and therefore equation 1 toward producing CO_{2(g)}, resulting in an increase of the rate of the dehydration of bicarbonate ion to CO₂(aq) catalyzed by carbonic anhydrase. The use of surfactants is expected to allow for a smaller size of the equipment necessary for the application and increasing the utility of the enzyme catalyzed process.

An aspect of the present invention is to include one or more surfactants in the CO₂ extraction processes and/or reactors of the present invention. The surfactant may be nonionic including semipolar and/or anionic and/or cationic and/or zwitterionic. Nonionic surfactant include but are not limited to alkyl polyethylene oxide, alkylphenol polyethylene oxide, copolymers of polyethylene oxide and polypropylene oxide (commercially called Poloxamers or Poloxamines), alkyl polyglucosides such as octyl glucoside, fatty alcohols such as cetyl alcohol and oleyl alcohol, polysorbates such as Tween 20 and Tween 80, dodecyl dimethylamine oxide. alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”). Anionic surfactants include but are not limited to perfluorooctanoate (PFOA or PFO), perfluorooctanesulfonate (PFOS), sodium dodecyl sulfate (SDS), ammonium lauryl sulfate and other alkyl sulfate salts, alkyl benzene sulfonate, linear alkylbenzenesulfonate, alphaolefinsulfonate, alkyl sulfate (fatty alcohol sulfate), alcohol ethoxysulfate, secondary alkanesulfonate, alpha-sulfo fatty acid methyl ester, alkyl-or alkenylsuccinic acid and soap. Cationic surfactants include, but are not limited to cetyl trimethylammonium bromide (CTAB) such as hexadecyl trimethyl ammonium bromide and other alkyltrimethylammonium salts, cetylpyridinium chloride (CPC), polyethoxylated tallow amine (POEA), benzalkonium chloride (BAC) and Benzethonium chloride (BZT). Zwitterionic surfactants include, but are not limited to dodecyl betaine, cocamidopropyl betaine and coco ampho glycinate. The surfactant may also contain PEG/VA polymers, ethoxylated (EO) or propoxylated (PO) polymers such as EO/PO polyethyleneimine, EO/PO polyamidoamine or EO/PO polycarboxylate (described in EP 1876227). Low-foaming nonionic surfactants are preferred for such application. Alkyl-capped non-ionic surfactants Cₙ(EO)ₘ is in this category. Also preferred are EO/PO block copolymers and certain silicone based surfactants or lubricants. Examples of commercially available surfactants are Ethox L-61, Ethox L62 and Ethox L64 (Ethox, Greenville, S.C. USA). The surfactant or surfactant/polymer mixture can typically be present in a level from 0.01% W/V to 5% W/V, preferably from 0.05% W/V to 2.5% W/V, more preferred from 0.1% W/V to 1% W/V. In a preferred embodiment, surfactant is present in the carrier liquid, most preferably surfactant is present in the desorption module(s). When surfactant is used in the extraction process, it is preferred to use membranes in the modules which do not leak in the presence of surfactant, preferably a PTFE membrane is used. Other preferred membranes include membranes made from polyimide, polyolefin (including polypropylene), polysulfone, silicone, or co-polymers and/or composites of these.

The desorption rate can also be increased by increasing the area of the gas-liquid interface. This can either be done by using a single desorption module with a larger surface area or by increasing the number of desorption modules. In an embodiment of the present invention the total surface area of the desorption module is different from the total surface area of the absorption module. In one embodiment of the present invention the total surface area of the desorption module(s) is at least 10% larger than the surface area of the absorption module, more preferably it is at least 20% larger than the surface area of the absorption module, even more preferably 30%, 50%, 70%, 100%, 200%, 300%, or 400%, larger than the surface area of the absorption module and most preferably it is at least 500% larger than the surface area of the absorption module. In another embodiment of the present invention the total surface area of the absorption module(s) is at least 10% larger than the surface area of the desorption module, more preferably it is at least 20% larger than the surface area of the desorption module, even more preferably 30%, 50%, 70%, 100%, 200%, 300%, or 400% times larger than the surface area of the desorption module and most preferably it is at least 500% larger than the surface area of the desorption module. The total gas-liquid surface area of the modules will depend on the amount of CO₂ that is expected to be captured by the reactor. For small scale capture such as in air revitalization in space suit or diving suits the surface area of the lab-scale reactors described in the present examples may suffice, whereas for extraction of CO₂ from a combustion process in, e.g., a power plant a much larger gas-liquid phase surface area will be needed. The surface area of each module will, therefore, need optimization depending on the application of the reactor. The modular design of the present invention allows for easy scale-up of the system.

The reactor of the present invention is suitable for extracting carbon dioxide from a gas phase, and can comprise any combination of the elements described above. Preferably, the reactor comprises the following elements: a) at least one absorption module (e.g., 7, FIG. 1) comprising at least one gas permeable membrane and a gas inlet zone (e.g., 14, FIG. 1) and a gas outlet zone (e.g., 15, FIG. 1); b) at least one desorption module (e.g., 8, FIG. 1) comprising at least one gas permeable membrane and a gas outlet zone (e.g., 19, FIG. 1); c) a carrier liquid; d) means for connecting the absorption module(s) and the desorption module(s) such that the carrier liquid from the absorption module(s) can be passed to the desorption module(s) from where it can be returned to the absorption module(s).

Preferably, the reactor of the present invention comprises one or more carbonic anhydrase.

In cases where there are multiple absorption modules, the outlet gas (scrubbed gas) from the first absorption module can be passed to a second absorption module in order to remove additional CO₂ which was not removed in the first absorption module. At the same time the carbon enriched carrier liquid from the first absorption module is passed on to a first desorption module and the carrier liquid from the second absorption module is passed on to a first desorption module or to a second desorption module. Examples of reactor configurations with multiple modules are shown in FIG. 3.

In order to enable process control of the carrier liquid volume, flow rate, and/or composition, the carrier liquid circulating continuously through the reactor can pass through one or more liquid reservoirs. These reservoirs can serve as a convenient point to add or remove carrier liquid, monitor and/or adjust the liquid pH and/or temperature, and make changes to the carrier liquid composition, such as adding more CO₂-absorbing chemicals, adding more carbonic anhydrase, and/or removing build-up of undesired contaminants, such as removing flocculated carrier liquid components by filtration or centrifugation, or such as inducing flocculation of undesired contaminants, such as build-up of precipitated solids, contaminant dissolved metals, or such as compounds formed by the combination of SOₓ or NOₓ with carrier liquid components, and removing these flocculated contaminants by filtration or centrifugation.

The reactor of the present invention may be used in a process for extraction of carbon dioxide from a carbon dioxide-containing gas.

A process of the present invention suitable for extracting CO₂ comprises the following steps: a) passing the gas through one or more membrane containing module(s) where carbon dioxide contained in the gas is absorbed by a carrier liquid passing through the module(s); b) passing the carrier liquid from the module(s) in step a) through one or more membrane containing module(s) where the absorbed carbon dioxide is allowed to desorb; c) returning the liquid from the module(s) in step b) to the module(s) in step a). Preferably, the pH of the carrier liquid passing from the desorption module is plus or minus one (±1) pH unit of the target pH before re-entry into the absorption module. The target pH of the carrier liquid (as measured at room temperature, e.g., 20-25° C.) is at least pH 6.5, more preferably above pH 7, more preferably above pH 7.5, more preferably above pH 8, even more preferably between pH 8 and 12, or within one of the other pH ranges mentioned above. In a further embodiment the carrier liquid is passed through at least one liquid reservoir. This may either be located after the desorption module and/or between the absorption and desorption modules.

Preferably, to maintain pH within the above mentioned pH ranges the carrier liquid comprises at least one buffering agent. Suitable buffering agents in the carrier liquid can be any buffering agent with a buffering range falling above pH 6.5, preferably above pH 7, more preferably above pH 7.5, more preferably within the range of pH 8 and pH 12, even more preferably in the range pH 8 and pH 10.5, without necessarily being capable of providing a stable pH within the whole range. A suitable buffering agent can, e.g., be selected from the group consisting of bicarbonate, phosphate, Tris; taurine, TABS, TAPS, hydrazine, HEPBS, CAPSO, ammonium hydroxide, AMP, AMPSO and AMDP. Furthermore, a suitable buffering agent can be a compound which, when combined with CO₂-absorbing amines of the present invention, forms a liquid that has a pH falling in the preferred ranges. The buffering agents may be combined into suitable mixtures of buffering agents. The most suitable concentration of buffering agent should be optimized from reactor to reactor, since it is dependent on several parameters such a CO₂ concentration in the feed gas, flow rate composition of the carrier liquid, pressure in the reactor modules, catalyst concentration (e.g., carbonic anhydrase), temperature, and surface area of the liquid-gas. A suitable buffer concentration could be between 20 mM and 2 M. Preferably, it is between 50 mM and 1.5 M, more preferably it is between 100 mM and 1 M. The present inventors have realized that the presence of bicarbonate ions in the carrier liquid, either alone or in combination with another buffering agent, facilitates the absorption of CO₂ from a mixed gas stream provided that the pH of the buffer is alkaline, preferably the pH of the buffer is maintained above pH 7.5, more preferably the pH is maintained between 8.5 and 12, more preferably between 8.5 and 11, more preferably between 8.5 and 10.5, more preferably between 9 and 10, even more preferably the pH is maintained between pH 9.2 and 9.5. Previously, the bicarbonate containing buffer system has been reported as being disadvantageous compared to a phosphate containing puffer system due to the pH variation in the system when CO₂ is captured in a carrier liquid (Trachtenberg et al., 2003, SAE international Conference on Environmental Systems Docket number 2003-01-2499). As described above the pH stability in the system can be ensured using the modular reactor system of the present invention. In a preferred embodiment of the present invention the buffering agent in the carrier liquid is bicarbonate, such as sodium bicarbonate, potassium bicarbonate, cesium bicarbonate or another suitable salt of the bicarbonate. When the pH in the carrier liquid is maintained above 8.5 the amount of carbonic anhydrase needed to extract CO₂ from the feed gas can be reduced between 5 to 100 times as compared to the reported amounts of 3 g/L.

A further parameter in the reactors of the present invention which can be optimized is the flow rate of the carrier liquid. Decreasing the liquid flow rate can increase the carrier liquid residence time in the desorption module which allows for more CO₂ to be extracted from the carrier liquid. Optimization of carrier liquid flow rate in each module can allow for increase mass transfer between liquid and gas phase. In order to facilitate different flow rates in the two modules extra carrier liquid reservoir can be added after the absorption module in which the carbon-rich liquid is collected and pumped through the desorption module with an additional liquid pump at a slower rate.

In the CO₂ extraction process of the present invention one or more carbonic anhydrase (EC 4.2.1.1) can be used as a CO₂ extraction catalyst. Preferably, one or more of the previously described carbonic anhydrases or a carbonic anhydrase describe in the section “Enzymes for the bioreactors” is used in the process. The amount of carbonic anhydrase is preferably below 2 g enzyme protein/L carrier liquid, more preferably it is below 1.5 g/L, even more preferably below 1 g/L, even more preferably below 0.6 g/L, even more preferably below 0.3 g/L, even more preferably below 0.1 g/L, even more preferably below 0.05 g/L, even more preferably below 0.01 g/L, and even more preferably below 0.005 g/L and even most preferably below 0.001 g/L. Because the rate of dehydration catalyzed by carbonic anhydrase is lower than the rate of hydration catalyzed by carbonic anhydrase, it is preferred that the amount of carbonic anhydrase in the dehydration module is higher than the amount of carbonic anhydrase in the hydration module. Preferably, the amount of carbonic anhydrase in the dehydration module is at least 0.005 g/L higher than in the hydration module, preferably it is at least 0.01 g/L higher than in the hydration module, preferably it is at least 0.05 g/L higher, more preferably it is 0.03 g/L higher and most preferably it is 0.1 g/L higher. The reactors of the present invention may also, as described above, comprise a carrier liquid with a chemical or physical solvent that have affinity toward CO₂ to facilitate the CO₂ extraction. Such chemicals can, e.g., constitute conventional CO₂ extraction technologies such as chemical absorption via amine-based solvents or aqueous ammonia or blends of such chemicals. Physical solvents can, e.g., be Selexol™ (Union Carbide) or water or glycerol or polyethylene glycol ethers, or polyethylene glycol dimethyl ether. Carbonic anhydrase may be combined with these conventional CO₂ extraction technologies. In PCT/US2008/052567 it has been shown that by adding carbonic anhydrase to a MEA solution the efficiency of the CO₂ hydration is significantly increased and the amount of carbonic anhydrase can be reduced at least 2 times. In a further embodiment of the present invention the carrier liquid comprises a carbonic anhydrase in combination with one or more carbon dioxide absorbing compound(s) such as amine-based compounds such as aqueous alkanolamines including monoethanolamine (MEA), diethanolamine (DEA), methyldiethanolamine (MDEA), 2-amino-2-methyl-1-propanol (AMP), 2-amino-2-hydroxymethyl-1,3-propanediol (AHPD), Tris or other primary, secondary, tertiary or hindered amine-based solvents such as piperazine and piperidine and derivatives of these, or polyethylene glycol ethers, or aqueous salts of amino acids such as glycine or derivatives of these such as taurine or other liquid absorbers such as aqueous NaOH, KOH, LiOH, carbonate or bicarbonate solutions at different ionic strengths or aqueous electrolyte solutions, or a blend of them or analogs or blends thereof. In conventional reactors, the concentration of alkanolamines is typically 15-30 weight percent. In conventional processes, free radical scavengers such as thiosulfate, sulfite, bisulfite, aromatic amines, and/or proprietary inhibitors, such as Fluor's EconAmine, are added to provide for using high amine concentration while reducing the risk of oxidation and corrosion. In the reactors and processes of the present invention, the concentration of alkanolamines is preferably below 15% (V/V), more preferably below 12%, 10%, 8%, 6%, 5%, 4%, 3%, 2%, 1%, 0.5%, 0.2% and most preferably below 0.1% (V/V).

In a further embodiment of the present invention, the carrier liquid comprising carbon dioxide absorbing compounds as described above is adjusted so that the pH of the resulting liquid is compatible with the active pH range of carbonic anhydrase enzyme.

In a further embodiment of the present invention, the carrier liquid comprises carbon dioxide absorbing compounds and carbonic anhydrase enzyme immobilized in one or more of the modules through which the carrier liquid passes and/or in a carrier liquid reservoir.

In a further embodiment of the present invention, the reactor comprises two or more different carbonic anhydrase enzymes. For example, one type of carbonic anhydrase enzyme is immobilized in the absorber module(s) and a different type of carbonic anhydrase is immobilized in the desorber module(s). In another non-limiting example, one type of carbonic anhydrase enzyme is immobilized in the absorber/desorber module(s) and/or in a carrier liquid reservoir and a different type of carbonic anhydrase is dissolved in the carrier liquid.

The process of the present invention for extracting carbon dioxide from a gas phase, can comprise any combination of the elements described above, including elements described in relation to bioreactors.

### Uses

The reactors and processes of the present invention may be used to extract CO₂ from CO₂ emission streams, e.g., from carbon-based or hydrocarbon-based combustion in electric generation power plants, or from flue gas stacks from such plants, industrial furnaces, stoves, ovens, or fireplaces or from airplane or car exhausts, in particular bioreactors comprising a heat-stable carbonic anhydrase is useful in these applications.

Other uses of the present invention is removal of CO₂ in the preparation of industrial gases such as acetylene (C₂H₂), carbon monoxide (CO), chlorine (Cl₂), hydrogen (H₂), methane (CH₄), nitrous oxide (N₂O), propane (C₃H₈), sulfur dioxide (SO₂), argon (Ar), nitrogen (N₂), and oxygen (O₂). Removal of CO₂ from a raw natural gas during the processing to natural gas is also contemplated. Removal of CO₂ from the raw natural gas will serve to enrich the methane (CH₄) content in the natural gas, thereby increasing the thermal units/m³. Raw natural gas is generally obtained from oil wells, gas wells, and condensate wells. Natural gas contains between 3 to 10% CO₂ when obtained from geological natural gas reservoirs by conventional methods. The reactor and process of the present invention can also be used to purify the natural gas such that it is substantially free of CO₂, e.g., such that the CO₂ content is below 1%, preferably below 0.5%, 0.2%, 0.1%, 0.05% and most preferably below 0.02%. In resemblance to the methane enrichment of natural gases, the present invention can also be used to enrich the methane content in biogases. Biogases will always contain a considerable degree of CO₂, since the bacteria used in the fermentation process produce methane (60-70%) and CO₂ (30-40%). Biogas production may be performed using mesophilic or thermophilic microorganisms. The process temperatures for mesophilic strains is approximately between 25° C. and 40° C., preferably between 30° C. and 35° C. In this temperature range the bioreactor may contain a carbonic anhydrase of bovine or human origin since there are no requirements to thermostability of the enzyme. Thermophilic strains allow the fermentation to occur at elevated temperatures, e.g., from 40° C. to 80° C., and preferably from 50° C. to 70° C. and even more preferably from 55° C. to 60° C. In such processes a bioreactor with a heat-stable carbonic anhydrase is particularly useful to remove CO₂ from the methane. The present invention can be used for reduction of the carbon dioxide content in a biogas, preferably the CO₂ content is reduced such that it constitutes less than 25%, more preferably less than 20%, 15%, 10%, 5%, 2%, 1%, 0.5% and most preferably less than 0.1%. In a preferred embodiment a bioreactor with a heat-stable carbonic anhydrase is used. Furthermore, the present invention may be applied in the production of syngas by removing the CO₂ generated by the gasification of a carbon containing fuel (e.g., methane or natural gas) thereby enriching the CO, H₂ content of the syngas. Where syngas production occurs at elevated temperatures the use of a heat-stable carbonic anhydrase is an advantage. The present invention can be used for the reduction of the carbon dioxide content in a syngas production. Preferably, the CO₂ content is reduced such that it constitutes less than 25%, more preferably less than 20%, 15%, 10%, 5%, 2%, 1%, 0.5% and most preferably less than 0.1%. In a preferred embodiment the carbonic anhydrase is heat-stable. Preferably, a heat-stable carbonic anhydrase for use in the bioreactor and CO₂ extraction processes of the present invention maintain activity at temperatures above 45° C., preferably above 50° C., more preferably above 55° C., more preferably above 60° C., even more preferably above 65° C., most preferably above 70° C., most preferably above 80° C., most preferably above 90° C., and even most preferably above 100° C. for at least 15 minutes, preferably for at least 2 hours, more preferably for at least 24 hours, more preferably for at least 7 days, even more preferably for at least 14 days, most preferably for at least 30 days, even most preferably for at least 50 days at the elevated temperature. The temperature stability of the carbonic anhydrase can be increased to some extent by way of formulation, e.g., by immobilization of the enzyme.

The reactors and processes of the present invention also find more unconventional applications such as in pilot cockpits, submarine vessels, aquatic gear, safety and firefighting gear and astronaut's space suits to keep breathing air free of toxic CO₂ levels. Other applications are to remove CO₂ from confined spaces, such as to reduce hazardous CO₂ levels from inside breweries and enclosed buildings carrying out fermentation, and from CO₂ sensitive environments like museums and libraries, to prevent excessive CO₂ from causing acid damage to books and artwork. A further alternative application is to remove CO₂ from ambient air, e.g. hot ambient air in a desert. In this case the carbonic anhydrase could for example be comprised in a reactor suitable for extracting CO₂ from ambient air as described in Stolaroff et al. 2008 Environ. Sci. Technol., 42, 2728-2735, such a reactor could for example take the form of an “artificial tree”.

Before the carbon dioxide-containing gas is processed in a reactor of the present invention, it may be purified to free it from contaminants which may interfere with the reactor functionality, e.g., by clotting outlets or membranes or diminishing the effectiveness of the carrier liquid or in case of bioreactors disturbing the enzymatic reaction. Gases/multiphase mixtures emitted from combustion processes, e.g., flue gases or exhausts, are preferably cleared of ash, particles, NOₓ and/or SOₓ (e.g., SO₂), before the gas/multiphase mixture is passed into the reactor. The raw natural gas from different geographic regions may have different compositions and separation requirements. Preferably, oil, condensate, water and natural gas liquids, if present in the raw natural gas, are removed prior to the extraction of CO₂ in a reactor of the present invention. The CO₂ from the raw natural gas may be extracted in the same process as the sulfur removal, or it may be extracted in a completely separate process. For bioreactors, the gas may at this point exceed the temperature optimum of the carbonic anhydrase present in the bioreactor, in this case some degree of cooling may be needed. Preferably, the reaction temperature is between 45° C. and 100° C., more preferably between 45° C. and 80° C., even more preferably between 45° C. and 60° C., and most preferably between 45° C. and 55° C. If CA-I or CA-II isolated from human or bovine erythrocytes is applied in the bioreactor the reaction temperature should not be above 37° C.

The CO₂ extracted by the process of the present invention can be used for a variety of purposes, such as for enhanced oil recovery, to form commodity carbonate salts, to separate the CO₂ for the purpose of sequestration, such as in CO₂-impermeable capped geological formations and/or in deep saline aquifers. Other applications are to extract CO₂ for the purpose of delivering the enriched CO₂ gas stream to enhance the growth of organisms that metabolize CO₂, such as plants, e.g., plants growing in greenhouses, or algae, e.g., algae growing in ponds or enclosed spaces, requiring delivery of CO₂ to maintain algae growth.

### Enzymes for the Bioreactors

The preferred enzyme for the bioreactors of the present invention is carbonic anhydrase.

Carbonic anhydrases (CA, EC 4.2.1.1, also termed carbonate dehydratases) catalyze the interconversion between carbon dioxide and bicarbonate [CO₂+H₂O⇄HCO₃⁻+H⁺]. The enzyme was discovered in bovine blood in 1933 (Meldrum and Roughton, 1933, J. Physiol. 80: 113-142) and has since been found widely distributed in nature in all domains of life from mammals, plant, fungi, bacteria and archaea. Carbonic anhydrase enzymes are categorized in three distinct classes called the alpha-, beta- and gamma-class, and potentially a fourth class, the delta-class. There are several sources of carbonic anhydrase, e.g., the mammalian alpha carbonic anhydrases CA-I or CA-II isolated from human or bovine erythrocytes which can be purchased commercially. US 2006/0257990 describes a variant of human carbonic anhydrase with increased thermostability. The gamma carbonic anhydrase, CAM, from Methanosarcina thermophila strain TM-1 (DSM 1825) is also well described (Alber and Ferry, 1994, Proc. Natl. Acad. Sci. USA 91: 6909-6913). WO 2008/095057 and U.S. Application no. 61220636 describe heat-stable alpha-carbonic anhydrase from bacteria. Any of these enzymes or blends of these enzymes may be used in the reactors and processes of the present invention. Preferred heat-stable carbonic anhydrases in the bioreactors and process of the present invention are SEQ ID NO: 2, 4, 6, 8, 10, 12, 14 or 16 from WO 2008/095057 (hereby incorporated by reference) or SEQ ID NO: 2 of U.S. application no. 61220636 (hereby incorporated by reference).

For certain applications, immobilization of the carbonic anhydrase may be preferred. An immobilized enzyme comprises two essential functions, namely the non-catalytic functions that are designed to aid separation (e.g., isolation of catalysts from the application environment, reuse of the catalysts and control of the process) and the catalytic functions that are designed to convert the target compounds (or substrates) to products within the time and space desired (Cao, Carrier-bound Immobilized Enzymes: Principles, Applications and Design, Wiley-VCH Verlag GmbH & Co. KGaA, Weinheim, Germany, 2005). When an enzyme is immobilized it is made insoluble to the target compounds (e.g., substrates) it aids converting and to the solvents used. An immobilized enzyme product can be separated from the application environment in order to facilitate its reuse, or to reduce the amount of enzyme needed in the application environment, or to use the enzyme in a process where substrate is continuously delivered and product is continuously removed from proximity to the enzyme, which, e.g., reduces the amount of enzyme needed per amount substrate converted. Furthermore, enzymes are often stabilized by immobilization which can allow the enzyme to operate longer in the application. A process involving immobilized enzymes is often continuous, which facilitates easy process control. The immobilized enzyme can be restrained by physical means, such as by entrapment of the enzyme in a space in such a way that the enzyme cannot move away from that space. For example, this can be done by entrapping the enzyme in a polymeric cage, wherein the physical dimensions of the enzyme are too large for it to pass between adjacent polymer molecules forming the cage. Entrapment can also be done by confining the enzyme behind membranes that allow smaller molecules to pass freely while retaining larger molecules, e.g., using semi permeable membranes or by inclusion in ultrafiltration systems using, e.g., hollow fiber modules, semi permeable membrane stacks, etc. Immobilization on porous carriers is also commonly used. This includes binding of the enzyme to the carrier, e.g., by adsorption, complex/ionic/covalent binding, or just simple absorption of soluble enzyme on the carrier and subsequent removal of solvent. Cross-linking of the enzyme can also be used as a means of immobilization. Immobilization of enzyme by inclusion into a carrier is also industrially applied. (Buchholz et al., Biocatalysts and Enzyme Technology, Wiley-VCH Verlag GmbH & Co. KGaA, Weinheim, Germany, 2005). Specific methods of immobilizing enzymes such as carbonic anhydrase include, but are not limited to, spraying of the enzyme together with a liquid medium comprising a polyfunctional amine and a liquid medium comprising a cross-linking agent onto a particulate porous carrier as described in WO 2007/036235 (hereby incorporated by reference), linking of carbonic anhydrase with a cross-linking agent (e.g., glutaraldehyde) to an ovalbumin layer which in turn adhere to an adhesive layer on a polymeric support as described in WO 2005/114417 (hereby incorporated by reference), or coupling of carbonic anhydrase to a silica carrier as described in U.S. Pat. No. 5,776,741 or to a silane, or a CNBr activated carrier surface such as glass, or co-polymerization of carbonic anhydrase with methacrylate on polymer beads as described in Bhattacharya et al., 2003, Biotechnol. Appl. Biochem. 38: 111-117 (hereby incorporated by reference). In an embodiment of the present invention carbonic anhydrase is immobilized on a matrix. The matrix may, e.g., be selected from the group beads, fabrics, fibers, hollow fibers, membranes, particulates, porous surfaces, rods, structured packing, and tubes. Specific examples of suitable matrices include alumina, bentonite, biopolymers, calcium carbonate, calcium phosphate gel, carbon, cellulose, ceramic supports, clay, collagen, glass, hydroxyapatite, ion-exchange resins, kaolin, nylon, phenolic polymers, polyaminostyrene, polyacrylamide, polypropylene, polymerhydrogels, sephadex, sepharose, silica gel, precipitated silica, and TEFLON-brand PTFE. In an embodiment of the present invention carbonic anhydrase is immobilized on a nylon matrix according to the techniques described in Methods in Enzymology, Volume XLIV (section in the chapter: Immobilized Enzymes, pages 118-134, edited by Klaus Mosbach, Academic Press, New York, 1976), hereby incorporated by reference.

The carbonic anhydrase to be included in a reactor or process may be stabilized in accordance with methods known in the art, e.g., by adding an antioxidant or reducing agent to limit oxidation of the carbonic anhydrase or it may be stabilized by adding polymers such as PVP, PVA, PEG, sugars, oligomers, polysaccharides or other suitable polymers known to be beneficial to the stability of polypeptides in solid or liquid compositions. A preservative, such as penicillin, virginiamycin, or Proxel™ (Arch Chemicals, Inc.), can be added to extend shelf life or performance in application by preventing microbial growth.

### EXAMPLES

### Methods

### Detection of Carbonic Anhydrase Activity

The test for the detection of carbonic anhydrase was described by Wilbur, 1948, J. Biol. Chem. 176: 147-154. The set up is based on the pH change of the assay mixture due to the formation of bicarbonate from carbon dioxide as given in equation 1: [CO₂+H₂O→HCO₃⁻+H⁺].

The activity assay used in this study was derived from the procedure of Chirica et al., 2001, Biochim. Biophys. Acta 1544(1-2): 55-63. A solution containing approximately 60 to 70 mM CO₂ was prepared by bubbling CO₂ into 100 ml distilled water using the tip of a syringe approximately 45 min to 1 h prior to the assay. The CO₂ solution was chilled in an ice-water bath. To test for the presence of carbonic anhydrase, 2 ml of 25 mM Tris, pH 8.3 (containing sufficient bromothymol blue to give a distinct and visible blue color) were added to two 13×100 mm test tubes chilled in an ice bath. To one tube, 10 to 50 microliters of the enzyme containing solution (e.g., culture broth or purified enzyme) was added, and an equivalent amount of buffer was added to the second tube to serve as a control. Using a 2 ml syringe and a long cannula, 2 ml of CO₂ solution was added very quickly and smoothly to the bottom of each tube. Simultaneously with the addition of the CO₂ solution, a stopwatch was started. The time required for the solution to change from blue to yellow was recorded (transition point of bromothymol blue is pH 6-7.6). The production of hydrogen ions during the CO₂ hydration reaction lowers the pH of the solution until the color transition point of the bromothymol blue is reached. The time required for the color change is inversely related to the quantity of carbonic anhydrase present in the sample. The tubes remain immersed in the ice bath for the duration of the assay for results to be reproducible. Typically, the uncatalyzed reaction (the control) takes approximately 2 min for the color change to occur, whereas the enzyme catalyzed reaction is complete in 5 to 15 sec, depending upon the amount of enzyme added. Detecting the color change is somewhat subjective but the error for triple measurements was in the range of 0 to 1 sec difference for the catalyzed reaction. One unit is defined after Wilbur [1 U=(1/t_{c})−(1/tᵤ)×1000] where U is units and t_{c} and tᵤ represent the time in seconds for the catalyzed and uncatalyzed reaction, respectively (Wilbur, 1948, J. Biol. Chem. 176: 147-154). These units are also termed Wilbur-Anderson units (WAU).

Kinetic Assay for Carbonic Anhydrase Activity with P-Nitrophenyl Acetate

Twenty microliters purified CA enzyme sample (diluted in 0.01% Triton X-100) was placed in the bottom of a micro-titer plate (MTP) well. The assay was started at room temperature by adding 200 microliters para-nitrophenol-acetate ((pNp-acetate, Sigma, N-8130) substrate solution in the MTP well. The substrate solution was prepared immediately before the assay by mixing 100 microliters pNP-acetate stock solution (50 mg/ml pNP-acetate in DMSO. Stored frozen) with 4500 microliters assay buffer (0.1 M Tris/HCl, pH 8). The increase in OD₄₀₅ was monitored. In the assay a buffer blind (20 microliters assay buffer instead of CA sample) was included. The difference in OD₄₀₅ increase between the sample and the buffer blind was a measure of the carbonic anhydrase activity (CA activity=ΔOD₄₀₅ (sample)−ΔOD₄₀₅ (buffer)).

### Example 1

### Extraction of CO₂ from a Mixed Gas Stream in a Modular Hollow Fiber Bioreactor

A lab-scale hollow fiber liquid membrane bioreactor (HFLMB) containing two modules one for hydration and one for dehydration was set up to selectively capture CO₂ from a gas stream resembling a flue gas.

### Hollow Fiber Liquid Membrane Bioreactor Set-Up

Porous hydrophobic hollow fiber membranes provide a high surface area of contact between the gas stream and carrier liquid. As a result they facilitate carbonation of a liquid or removal of CO₂ from a liquid. The reactor consisted of two polypropylene hollow fiber membrane modules. The hydration module consisted of 2300 parallel hollow fibers with 0.18 m² active surface area and average pore size of 0.01×0.04 micrometer (MiniModule® 1.0×5.5 part # G543, Membrana, Charlotte, N.C., USA). The de-hydration module was larger and consisted of 7400 parallel hollow fibers with 0.58 m² active surface area and average pore size of 0.01×0.04 micrometer (MiniModule® 1.7×5.5 part # and G542, Membrana, Charlotte, N.C., USA). These membranes are easy to scale-up to industrial scale and have been used in industry for wastewater treatment and beverage carbonation. A schematic drawing of the bioreactor set-up is shown in FIG. 1. Briefly described the set-up was as follows: a carrier liquid (heavy black line in FIG. 1) containing the carbonic anhydrase passed through two sequential modules (7, 8, FIG. 1) using a positive displacement pump (5, FIG. 1) and recycled back to a reservoir (4, FIG. 1). Carrier liquid passed through the lumens of the hollow fibers (8, FIG. 3) in each module in this design. The liquid flow rate was set to about 4 ml/min. A pH probe in the reservoir monitored the pH throughout the experiment. A CO₂-containing mixed gas stream containing a mixture of 15% CO₂ (9 CCM) and 85% N₂ (51 CCM) entered the shell side of the hydration module (7, 14 FIG. 1) counter-currently and the scrubbed stream exits the module (7, 15, FIG. 1). A sweep stream of nitrogen passed through the dehydration module (8, 18, 19, FIG. 1) allowing CO₂ stripping from the carrier liquid. The flow rate of the sweep gas was adjusted such that a constant pH of the carrier liquid (pH=9) in the reservoir was maintained (steady state). The sweep flow rate was adjusted carefully. Too high flow rate of sweep gas resulted in gradual increase of the pH of the carrier liquid in the reservoir, whereas too low flow rate lead to gradual decrease of the pH of the carrier liquid.

Two mass flow controllers (3, FIG. 1) were used to mix nitrogen and carbon dioxide with consistent concentration throughout the experiments. Mass flow meters (11, FIG. 1) were used to monitor the flow of the scrubbed gas, CO₂-containing mixed gas and the sweep gas throughout the reactor run. The gas and liquid flows and pressures are adjusted in a way to avoid liquid entering the gas phase and to avoid gas bubbles in the liquid phase of the modules.

When operating the reactor at a higher temperature (e.g., 50° C.), both hydration and dehydration modules were wrapped with heating tapes and were insulated via insulation tapes. Thermocouples were used on the outside of each module to maintain the temperature of the modules at the target temperature via temperature controllers. The carrier liquid in the reservoir was stirred and maintained at the target temperature via a magnetic hot plate equipped with a thermocouple.

### Carrier Liquid

A mixture of 1 M sodium bicarbonate and 1 M sodium hydroxide solution with pH=9 was used as a carrier liquid control. Then, 0.03 mg/mL of an alpha-carbonic anhydrase (CA) enzyme protein originating from Bacillus clausii KSM-K16 (uniprot acc. No. Q5WD44) was added to the membrane reservoir. The volume of the liquid in the reservoir was maintained at 300 mL to compensate for evaporation during run time. The pH was continuously maintained at 9 by controlling the flow of the sweep gas in the dehydration module. The temperature was either room temperature or 50° C.

### Gas Chromatography Method

The amount of CO₂ in the CO₂-containing mixed gas (inlet gas) and scrubbed gas (outlet gas) were analyzed by GC. Data were collected via injections of samples to GC. At least four samples were collected per day, calculating an average for each day for a period of 10 days. A Shimadzu 2010 gas chromatograph with a thermal conductivity detector and a gas sampling valve was used for CO₂ concentration measurement. A capillary Carboxen Plot 1010 column was used to detect nitrogen and carbon dioxide. The column was heated isothermally for 7 minutes at 35° C., the temperature was increased with 20° C./min rate to 200° C. and it was maintained at 200° C. for 2 minutes. Injector and detector temperatures were maintained at 230° C. Column flow is 1 ml/min, split ratio 10 to 1 and carrier gas was helium. Nitrogen and carbon dioxide peaks were detected at retention times 6.4 and 15.3 minutes, respectively. The CO₂ peak was calibrated using three carbon dioxide standards with 0.1%, 1% and 10% CO₂ in nitrogen purchased from Scott Specialty gases (Pennsylvania, USA).

### Results

Table 1 shows the data collected during 10 days run time of the reactor. Each data point is the average of the measurements made each day during run time of ten days at room temperature. No loss of carbonic anhydrase activity was observed during the run time since no decrease in performance in the bioreactor could be observed over time. This was confirmed by measuring the activity of carrier liquid using the Wilbur Anderson based assay described under the methods “Detection of Carbonic Anhydrase Activity” above.

The results indicate that 0.03 mg/mL carbonic anhydrase enzyme protein significantly increases the HFLMB efficiency of CO₂ removal to about 80% compared to a control run at the same conditions without enzyme (˜24%). Also, it was shown that during the run time of 10 days at room temperature the enzyme maintains its maximal activity through repeated use, and the pH of the carrier liquid could be maintained at 9±0.05 by the use of a sweep gas.

TABLE 1
Performance of bioreactor during 10 day
continuous run at room temperature

| | % CO₂ | % CO₂ |
|---|---|---|
| | scrubbed gas | Removed |
| Run time (days) | (avg) | (avg) |
| 0 | 3.16 | 78.75 |
| 1 | 3.56 | 76.00 |
| 2 | 3.15 | 78.81 |
| 3 | 2.85 | 80.80 |
| 4 | 2.77 | 81.34 |
| 7 | 2.70 | 81.79 |
| 8 | 2.71 | 81.74 |
| 9 | 2.65 | 82.14 |
| 10 | 2.70 | 81.81 |
| Scrubbed gas avg. | 2.99 | 79.89 |
| (0.03 g/L CA e.p.) | | |
| Scrubbed gas avg. control | 11.27 | 24.12 |
| (no CA) | | |
| Feed gas avg. | 14.85 | N/A |

### Example 2

### Extraction of CO₂ from a Mixed Gas Stream in a Modular Hollow Fiber Bioreactor at 50° C.

This experiment was carried out essentially as described in example 1, with the minor changes described below. The sweep stream of nitrogen passed through the dehydration module was set at 60 CCM, and a bubbler was added to the reservoir (4, FIG. 1) to continuously bubble nitrogen for pH adjustment of the carrier liquid in the reservoir. The flow rate of the additional sweep gas in the bubbler was adjusted such that a constant pH of the carrier liquid (pH=9) in the reservoir was maintained (steady state). The carrier liquid was composed of a mixture of 0.5 M sodium bicarbonate and 0.5 M sodium hydroxide solution with pH=9 and maintained at 50° C.

### Results

Table 2 shows the data collected during 11 days run time of the reactor at 50° C. Each data point is the average of the measurements made each day during run time of eleven days at room temperature. Measuring the activity of carrier liquid using the Wilbur Anderson based assay described under the methods “Detection of Carbonic Anhydrase Activity” above showed some loss of carbonic anhydrase activity was observed during the run. However, no decrease in performance in the bioreactor could be observed over time. The results indicate that 0.03 mg/mL carbonic anhydrase enzyme protein significantly increases the HFLMB efficiency of CO₂ removal to about ˜75% compared to a control run at the same conditions without enzyme (˜40%). Also, it was shown that during the run time of 11 days at 50° C. the enzyme maintains its maximal performance in the reactor through repeated use.

TABLE 2
Performance of bioreactor during 11 day continuous run at 50° C.

| | | | WA |
|---|---|---|---|
| | % CO₂ | % CO₂ | residual |
| | scrubbed gas | removed | activity |
| Run time (days) | (avg) | (avg) | (%) |
| 0 | 3.7 | 74.4 | 100.0 |
| 3 | n.d. | n.d. | 111.5 |
| 4 | 3.1 | 78.4 | n.d. |
| 6 | n.d. | n.d. | 81.3 |
| 7 | 3.9 | 72.8 | 100.0 |
| 10 | 3.9 | 72.9 | 81.3 |
| 11 | 3.7 | 74.3 | 81.3 |
| Scrubbed gas avg. | 3.7 | 74.6 | n.d. |
| (0.03 g/L CA e.p.) | | | |
| Scrubbed gas avg. Control | 8.4 | 39.8 | n.d. |
| (no CA at 50° C.) | | | |

n.d. = not determined

### Example 3

### Desorption of CO₂ from a CO₂-Rich Carrier Liquid in a Hollow Fiber Membrane Module

A lab-scale bioreactor containing one hollow fiber membrane module for desorption was set up to desorb or extract CO₂ from a CO₂-rich carrier liquid such as 1M sodium bicarbonate at pH 8.

### Bioreactor Set-Up

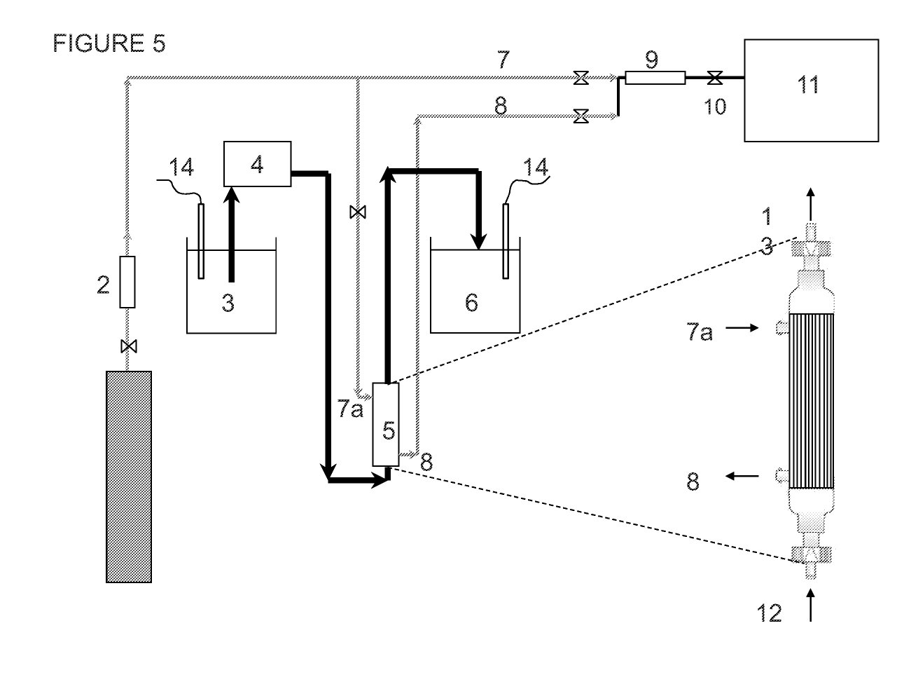

The reactor consisted of a polypropylene hollow fiber membrane module for desorption. The desorption module consisted of 2300 parallel hollow fibers with 0.18 m² active surface area and average pore size of 0.01×0.04 micrometer (MiniModule® 1.0×5.5 part # G543, Membrana, Charlotte, N.C., USA). These membranes are easy to scale-up to industrial scale and have been used in industry for wastewater treatment degassing and beverage carbonation. A schematic drawing of the bioreactor set-up is shown in FIG. 5. Briefly described the set-up was as follows: a carrier liquid (heavy black line in FIG. 5) containing the carbonic anhydrase passed through the lumens of the hollow fibers in the desorption module (5, FIG. 5) using a positive displacement pump (4, FIG. 5) going to a waste container (6, FIG. 5). The liquid flow rate was set to about 4 ml/min. pH probes in the carrier liquid reservoir and waste container monitored the pH throughout the experiment (14, FIG. 5). A CO₂-free sweep gas stream (7, FIG. 5) of nitrogen (60 CCM) entered the shell side of the desorption module at the inlet (7a, FIG. 5) counter-currently allowing CO₂ removal from the carrier liquid. The sweep stream containing CO₂ (8, FIG. 5) exited the module at the outlet.

A mass flow controller was used to maintain a constant flow in the sweep stream (2, FIG. 5). Also a mass flow meter (9, FIG. 5) were used to monitor the flow of the sweep stream containing CO₂. The gas and liquid flows and pressures were adjusted in a way to avoid liquid entering the gas phase of the module and to avoid gas bubbles in the liquid phase of the module.

The carrier liquid in the reservoir was stirred at room temperature via a magnetic stir plate.

### Carrier Liquid

A freshly prepared 1 M sodium bicarbonate solution pH 8 was used as a CO₂-rich carrier liquid control. Once all the data for the control runs without the enzyme was collected, another fresh 1M sodium bicarbonate solution containing 0.03 mg/mL of an alpha-carbonic anhydrase (CA) enzyme protein originating from Bacillus clausii KSM-K16 (uniprot acc. No. Q5WD44) was prepared as carrier liquid. The pH of the carrier liquid reservoir and waste solution was monitored during the experiment and the temperature was maintained at room temperature.

### Gas Chromatography Method

The amount of CO₂ in the sweep stream (inlet gas) and sweep stream containing CO₂ (outlet gas) were analyzed by GC. Data were collected via injections of samples to GC. At least three samples were collected during the run, calculating an average for a period of several hours. A Shimadzu 2010 gas chromatograph with a thermal conductivity detector and a gas sampling valve was used for CO₂ concentration measurement. A capillary Carboxen Plot 1010 column was used to detect nitrogen and carbon dioxide. The column was heated isothermally for 7 minutes at 35° C., the temperature was increased with 20° C./min rate to 200° C. and it was maintained at 200° C. for 2 minutes. Injector and detector temperatures were maintained at 230° C. Column flow is 1 ml/min, split ratio 10 to 1 and carrier gas was helium. Nitrogen and carbon dioxide peaks were detected at retention times 6.4 and 15.3 minutes, respectively. The CO₂ peak was calibrated using three carbon dioxide standards with 0.1%, 1% and 10% CO₂ in nitrogen purchased from Scott Specialty gases (Pennsylvania, USA).

### Results

Table 3 shows the data collected during the run time of the reactor. Each data point is the average measurement from three injections during run time at room temperature. Passing the carrier solution without enzyme through contactor raises the pH of solution from 8.0 to 8.3, the CO₂ content of the enriched gas was measured to be 3.3%. When 0.03 mg/mL carbonic anhydrase enzyme protein was in the carrier liquid a pH shift from 8.1 to 8.8 was observed and the CO₂ content of the enriched gas was about 10%. The results indicate that 0.03 mg/mL carbonic anhydrase enzyme protein significantly increases the CO₂ extraction efficiency of the carrier liquid. It is important to note that during runtime of the reactor the pH of carrier liquid reservoir raised from 8 to 8.1 for control solution within 75 minutes. When carbonic anhydrase was in the carrier liquid the pH raised from 8 to 8.2 within same time frame. The raise of pH in reservoir is due to partial dehydration of CO₂-rich carrier liquid in the reservoir before passing through the reactor. This raise in pH as expected is faster when carbonic anhydrase is present in the carrier solution.

TABLE 3
Performance at room temperature

| | % CO₂ in sweep | pH of | pH of |
|---|---|---|---|
| | gas exiting | carrier liquid | waste |
| | the module | reservoir | container |
| Carrier Liquid | (avg) | (avg) | (avg) |
| Water | 0.0 | n.d | n.d |
| 1M NaHCO3 | 3.3 | 8.0 | 8.3 |
| 1M NaHCO3 + | 10.0 | 8.1 | 8.8 |
| 0.03 g/L CA | | | |

### Example 4

### Effect of Surfactant on Dehydration Rate

The following experiment was performed to demonstrate the ability of surfactant to increase the rate of mass transfer of CO₂ from the aqueous to the gas phase.

When CO₂ is removed from the solution the pH will increase. The solution can be titrated via an acid such as hydrochloric acid during the dehydration reaction to maintain the pH constant. A pH stat auto-titrator allows measuring the rate of addition of acid during titration to maintain the pH of the solution at constant level. The rate of HCl addition directly corresponds to the rate of dehydration or pH increase. Therefore, the pH stat assay described below allows for the measurement of carbonic anhydrase catalyzed production of CO₂ from a bicarbonate solution.

### pH Stat Assay:

A 0.5 M sodium bicarbonate solution was freshly prepared (less than 4 hours old) before starting the experiment. Prior to the experiment the bicarbonate solution was kept covered to reduce any effect that may occur due to spontaneous conversion of bicarbonate to carbon dioxide. The experiment was performed using 35 ml of the NaHCO₃ solution at room temperature in a 50 ml Pyrex beaker. The assay was run with alpha-carbonic anhydrase enzyme obtained from Bacillus clausii-KSM-K16 (Uniprot acc. No. Q5WD44 described in WO 2008/095057) at different concentrations in the range of 0 to 0.08 mg enzyme protein/ml (0, 0.02, 0.04, 0.05, 0.06, and 0.08 mg enzyme protein/nil) and 0.1% W/V of one of the surfactants Ethox L-64, Ethox L-62 and Ethox L61 (Ethox, Greenville, S.C., USA) or no surfactant. The pH stat (718 STAT Titrino) which was connected to the Pyrex beaker was started immediately after addition of the enzyme (last ingredient to be added) and the addition of titrant (0.129M HCl) was recorded for 5 minutes. The pH stat was programmed to maintain the pH at 8 using the following settings:

| | | |
|---|---|---|
| Dynamics | 1.00 | |
| Max rate | 10.0 | ml/min |
| Min rate | 25 | ul/min |
| Start volume of titrant | 0.5 | ml* |
| Start time | 120 | sec |
| Stop time | 300 | sec |
| Stop volume | 5.0 | ml |

*0.5 ml of tritant was added at the beginning of the titration in order to bring the pH closer to 8.0 prior to starting the assay.

To facilitate the release of CO₂(g) during titration, a 7×20 mm stir bar was added to the beaker with a medium stirring rate.

### Results

It was shown that using a surfactant can enhance the apparent dehydration rate of sodium bicarbonate solution to carbon dioxide. Specifically, Table 4 shows that addition of surfactants caused a general increase in the rate of HCl added to the solution as a result of the pH rise. Importantly, it has been shown that addition of enzyme in the presence of the surfactant can enhance this effect. The increase in dehydration rate is believed to be due to the effect of surfactant in reducing surface tension of the solution and aiding mass transfer of carbon dioxide from liquid to gas phase.

TABLE 4
Effect of 0.1% w/v non-ionic non-foaming surfactant on
the apparent rate of dehydration of 0.5M NaHCO₃ solution
containing different amounts of carbonic anhydrase.

| Enzyme | | | | |
|---|---|---|---|---|
| concentration | HCl added by pH stat (mL/min) | | | |
| (mg/mL) | No Surfactant | Ethox L61 | Ethox L62 | Ethox L64 |
| 0 | 0.104 | 0.1809 | 0.1365 | 0.17995 |
| 0.02 | 0.15925 | 0.2462 | 0.23685 | 0.25585 |
| 0.04 | 0.19425 | 0.2848 | 0.2142 | 0.29135 |
| 0.05 | N/A | 0.2783 | 0.23295 | 0.2328 |
| 0.06 | 0.1673 | N/A | N/A | 0.2341 |
| 0.08 | 0.15765 | N/A | N/A | N/A |

### EMBODIMENTS OF THE INVENTION

1. A process for extraction of carbon dioxide from a carbon dioxide-containing gas comprising:
a) passing the gas through one or more membrane containing module(s) wherein carbon dioxide contained in the gas is absorbed by a carrier liquid passing through the module(s);
b) passing the carrier liquid from the module(s) in step a) through one or more membrane containing module(s) wherein the absorbed carbon dioxide is allowed to desorb; and
c) returning the liquid from the module(s) in step b) to the module(s) in step a);
d) wherein one or more carbonic anhydrase (EC 4.2.1.1) is used in the process.
   2. The process according to embodiment 1, wherein the pH of the carrier liquid after step b) is +/− one pH unit of the target pH before re-entry into the module(s) in step a).
   3. The process according to embodiment 1 or 2, further comprising passing the carrier liquid through at least one liquid reservoir after step a) and/or after step b).
   4. The process according to embodiment 3, wherein the pH of the carrier liquid after passing through the liquid reservoir(s) after step b) is +/− one pH unit of the target pH before re-entry into the module(s) in step a).
   5. The process according to any one of the preceding embodiments, wherein the carbonic anhydrase(s) is present in the module(s) of step a) and/or the module(s) of step b) and/or the liquid reservoir(s).
   6. The process according to any one of the preceding embodiments, wherein the carbonic anhydrase(s) is in solution in the carrier liquid.
   7. The process according to any one of embodiments 1 to 5, wherein the carbonic anhydrase(s) is immobilized on the membrane in the module(s) of step a) and/or the module(s) of step b) and/or in the interior of the liquid reservoir(s).
   8. The process according to any one of embodiments 1 to 5, wherein the carbonic anhydrase(s) is immobilized on a solid support contained within or entrapped within at least one of the module(s) of step a) and/or the module(s) of step b) and/or in the liquid reservoir(s).
   9. The process according to any one of the preceding embodiments, wherein the membrane containing modules are selected from the group consisting of a hollow fiber module a flat sheet membrane stack module and a spiral-wound membrane module.
   10. The process according to any one of the preceding embodiments, wherein the module(s) of step b) is supplied with a sweep stream.
   11. The process according to any one of the preceding embodiments, wherein the module(s) of step b) has a total surface area that is different than the surface area of the module(s) of step a).
   12. The process according to embodiment 11, wherein the module(s) of step b) has a total surface area that is larger than the surface area of the module(s) of step a).
   13. The process according to any one of the preceding embodiments, wherein the temperature in the module(s) of step b) is different than in the module(s) of step a).
   14. The process according to embodiment 13, wherein the temperature in the module(s) of step b) is at least 20° C. higher than the temperature in the module(s) of step a).
   15. The process according to any one of the preceding embodiments, wherein the module(s) of step b) is supplied with a low pressure steam.
   16. The process according to any one of the preceding embodiments, wherein the pressure in the module(s) of step b) is at least 35 kPa lower than the pressure in the module(s) of step a).
   17. The process according to any one of the preceding embodiments, wherein the pH of the carrier liquid before step a) is pH 8 or above.
   18. The process according to any one of the preceding embodiments, wherein the carrier liquid comprises water and/or bicarbonate, and/or amine-based CO₂ absorber chemicals, and/or alkaline salts, and or glycerol, and/or polyethylene glycol, and/or polyethylene glycol ethers.
   19. The process according to embodiment 18, wherein the carrier liquid comprises bicarbonate.
   20. The process according to any of the preceding embodiments, where surfactant is used in the process.
   21. The process according to embodiment 20, wherein the surfactant is present in the modules of step b).
   22. A reactor for extracting carbon dioxide from a gas phase, wherein said reactor comprises the following elements:
a) at least one absorption module comprising at least one gas permeable membrane and a gas inlet zone and a gas outlet zone;
b) at least one desorption module comprising at least one gas permeable membrane and a gas outlet zone;
c) a carrier liquid;
d) one or more carbonic anhydrase (EC 4.2.1.1); and
e) means for connecting the absorption module(s) and the desorption module(s) such that the carrier liquid can circulate from the absorption module(s) to the desorption module(s) and be returned to the absorption module(s).
   23. The reactor according to embodiment 22, which further comprises means for regulating pH in the carrier liquid.
   24. The reactor according to embodiment 22 or 23, which further comprises at least one liquid reservoir connected to the absorption and/or desorption module(s).
   25. The reactor according to any one of embodiments 22 to 24, wherein the carbonic anhydrase(s) is present in the absorption and/or desorption module(s) and/or in the liquid reservoir(s).
   26. The reactor according to any one of embodiments 22 to 25, wherein the carbonic anhydrase(s) is in solution in the carrier liquid.
   27. The reactor according to any one of embodiments 22 to 25, wherein the carbonic anhydrase(s) is immobilized on a membrane surface in the absorption and/or desorption module(s) and/or in the interior of the liquid reservoir(s).
   28. The reactor according to any one of embodiments 22 to 25, wherein the carbonic anhydrase(s) is immobilized on a solid support contained within or entrapped within at least one of the absorption and/or desorption module(s) and/or in the liquid reservoir(s).
   29. The reactor according to any one of embodiments 22 to 28, wherein the gas permeable membrane containing modules are selected from the group consisting of a hollow fiber membrane module, a flat sheet membrane stack module, and a spiral-wound liquid membrane module.
   30. The reactor according to any one of embodiments 22 to 29, wherein the desorption module(s) has a gas inlet zone.
   31. The reactor according to any one of embodiments 22 to 30, wherein the desorption module(s) has a total surface area that is different than the surface area of the absorption module(s).
   32. The reactor according to embodiment 31, wherein the desorption module(s) has a total surface area that is larger than the surface area of the absorption module(s).
   33. The reactor according to any one of embodiments 22 to 32, wherein the desorption module(s) and/or absorption module(s) is connected to means for heating and/or cooling the module(s).
   34. The reactor according to any one of embodiments 22 to 33, wherein the desorption module(s) is connected to a source for a low pressure steam.
   35. The reactor according to any one of embodiments 22 to 34, wherein the desorption module(s) is connected to a source for reducing the pressure.
   36. The reactor according to any one of embodiments 22 to 35, wherein the carrier liquid has a pH between 8 to 12.
   37. The reactor according to any one of embodiments 22 to 36, where the carrier liquid comprises water, and/or bicarbonate, and/or amine-based CO₂ absorber chemicals, and/or alkaline salts, and or glycerol, and/or polyethylene glycol, and/or polyethylene glycol ethers.
   38. The reactor according to embodiment 37, wherein the carrier liquid comprises bicarbonate.
   39. The reactor according to any of embodiments 22 to 38, where surfactant is present in the reactor.
   40. The process according to embodiment 39, wherein the surfactant is present in the desorption module(s).

1. A process for extraction of carbon dioxide from a carbon dioxide-containing gas comprising:
a) passing the gas through one or more membrane containing module(s) wherein carbon dioxide contained in the gas is absorbed by a carrier liquid passing through the module(s);
b) passing the carrier liquid from the module(s) in step a) through one or more membrane containing module(s) wherein the absorbed carbon dioxide is allowed to desorb; and
c) returning the liquid from the module(s) in step b) to the module(s) in step a);
wherein one or more carbonic anhydrase (EC 4.2.1.1) is used in the process.

2. The process according to claim 1, further comprising passing the carrier liquid through at least one liquid reservoir after step a) and/or after step b).

3. The process according to claim 1, wherein the carbonic anhydrase(s) is immobilized on the membrane in the module(s) of step a) and/or the module(s) of step b) and/or in the interior of the liquid reservoir(s).

4. The process according to claim 1, wherein the module(s) of step b) is supplied with a sweep stream.

5. The process according to claim 1, wherein the module(s) of step b) has a total surface area that is different than the surface area of the module(s) of step a).

6. The process according to claim 1, wherein the temperature in the module(s) of step b) is different than in the module(s) of step a).

7. The process according to claim 1, wherein the pressure in the module(s) of step b) is at least 35 kPa lower than the pressure in the module(s) of step a).

8. The process according to claim 1, where surfactant is used in the process.

9. A reactor for extracting carbon dioxide from a gas phase, wherein said reactor comprises the following elements:
a) at least one absorption module comprising at least one gas permeable membrane and a gas inlet zone and a gas outlet zone;
b) at least one desorption module comprising at least one gas permeable membrane and a gas outlet zone;
c) a carrier liquid;
d) one or more carbonic anhydrase (EC 4.2.1.1); and
e) means for connecting the absorption module(s) and the desorption module(s) such that the carrier liquid can circulate from the absorption module(s) to the desorption module(s) and be returned to the absorption module(s).

10. The reactor according to claim 9, which further comprises means for regulating pH in the carrier liquid.

11. The reactor according to claim 9, which further comprises at least one liquid reservoir connected to the absorption and/or desorption module(s).

12. The reactor according to claim 9, wherein the desorption module(s) has a gas inlet zone.

13. The reactor according to claim 9, wherein the desorption module(s) has a total surface area that is different than the surface area of the absorption module(s).

14. The reactor according to claim 9, wherein the desorption module(s) and/or absorption module(s) is connected to means for heating and/or cooling the module(s).

15. The reactor according to claim 9, wherein the desorption module(s) is connected to a source for reducing the pressure.

16. The reactor according to claim 9, where surfactant is present in the reactor.
